(19) United States
(12) Patent Application Publication
(10) Pub. No.: US 2021/0163872 A1
(43) Pub. Date: Jun. 3, 2021

(54) Cell Reprogramming Therapy

(71) Applicants: Rutgers, The State University of New Jersey, New Brunswick, NJ (US); The General Hospital Corporation, Boston, MA (US)

(72) Inventors: Biju Parekkadan, Piscataway, NJ (US); Matthew Li, Somerville, MA (US)

(21) Appl. No.: 16/961,098

(22) PCT Filed: Jan. 11, 2019
(86) PCT No.: PCT/US2019/013367

§ 371 Date: Jul. 9, 2020

Related U.S. Application Data
(60) Provisional application No. 62/616,930, filed on Jan. 12, 2018.

(51) Int. Cl. C12M 1/42; C12M 1/12; C12M 1/00; C12N 15/86; C12N 5/0783; C12N 5/0786

(52) U.S. Cl. CPC C12M 35/08; C12N 2740/15043; C12M 25/02; C12M 29/10; C12N 15/86; C12M 29/14; C12N 5/0636; C12N 5/0645; C12N 2521/00; C12N 2510/00; C12N 2502/1394; C12N 2502/1358; C12N 2502/1323; C12N 2502/1114; C12M 25/10

(57) ABSTRACT

Systems and methods for the dynamic co-culturing of two cell populations are provided. The system includes a barrier configured to physically separate a stimulator cell population from a responder cell population disposed within a container. The barrier is permeable to the secreted factors of at least one of the cell populations. The responder cell population can thereby be altered by exposure to the secreted factors to produce a population of reprogrammed cells that includes biomolecules (e.g., nucleic acids) originating from the stimulator cell population and/or that exhibits one or more additional or modified functional activities than a parental population of the reprogrammed cells.

### RELATED APPLICATION

This application claims the benefit of U.S. Provisional Application No. 62/616,930, filed on Jan. 12, 2018. The entire teachings of the above application are incorporated herein by reference.

### GOVERNMENT SUPPORT

This invention was made with government support under Grant Nos. R01EB012521 and T32EB016652-01A1 awarded by the National Institutes of Health. The government has certain rights in the invention.

### BACKGROUND

Hematopoietic cells are often used in cell therapies due to their ability to reconstitute blood cells in the human body (Lorenz et al., 1951). Bone marrow, peripheral blood-mobilized hematopoietic stem cells (HSCs), umbilical cord HSCs, red blood cells, platelets, and leukocytes are viable cell sources that are harvested from patients and prepared for intravenous administration to restore hematopoiesis. As hematopoietic cell therapies continue to expand, there exists a need for improved bioprocessing systems that can be used for the ex vivo maintenance and engineering of hematopoietic cells for clinical applications.

### SUMMARY

Systems and methods are provided for dynamic co-culturing of stimulator and responder cells, such as, for example, fibroblast stimulator cells and HSC responder cells, to support, e.g., ex vivo expansion and bioprocessing of therapeutic cells.

In some embodiments, a co-culture system comprises a responder cell population and a stimulator cell population disposed within a container. A barrier is configured to physically separate the responder cell population from the stimulator cell population, the barrier being permeable to secreted factors of the stimulator cell population. The system further includes a fluidic flow driver configured to induce a flow of a liquid suspension comprising at least one of the responder and stimulator cell populations through the container.

In other embodiments, a method of reprogramming cells is provided. The method includes exposing a responder cell population to the secreted factors of a stimulator cell population. The responder and stimulator cell populations are disposed within a container, and the secreted factors perfuse across a barrier separating the responder and stimulator cell populations in the container such that the responder cell population is altered following exposure to the secreted factors. The method further includes inducing a flow of a cell culture medium comprising at least one of the responder and stimulator cell populations in the container.

In further embodiments, a composition is provided, which includes a population of reprogrammed cells. The reprogrammed cells include nucleic acids originating from a different cell population. The reprogrammed cells also exhibit one or more additional or modified functional activities than a parental population of the reprogrammed cells. The composition can be administered to a patient in need thereof.

In other embodiments, a method of administering a composition of reprogrammed cells is provided to a patient in need thereof, such as, for example, a patient having an autoimmune disorder, an inflammatory disease, or a transplant. The composition may be delivered by intravenous administration or local administration and may include a dose of reprogrammed cells of about 5 million to about 1 billion.

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1 is a schematic of a dynamic co-culture system.

FIGS. 2A-2C illustrate the results of a 3T3-supported 2D short-term co-culture enriched for LSKs. Analysis was performed on whole bone marrow cells (BMCs) at a ratio of 1 stromal cell:10 BMCs. Cells were cultured in a non-contact dependent manner for 72 hours. FIG. 2A is a graph of cell yields at the end of the 72 hour culture period. FIG. 2B is a graph of Lineage^{negative} Sca^{positive} cKit^{positive} (LSK) proportions. FIG. 2C is an illustration of representative gates of LSK cells from the Lineage^{negative} population. The data is representative of three biological replicates. All values are means+standard deviation. The * indicates p-values compared to BM alone. *p<0.05, **p<0.01, and ***p<0.0001.

FIGS. 3A-3D illustrate a slow flow culture of human hematopoietic cells in a microreactor. Analysis was performed on BMCs after 1 hour of device seeding. FIG. 3A is a schematic of a slow flow culture system in which gas-exchange cell bags containing whole BMCs were connected to the intra-capillary space of a hollow fiber microreactor, which in turn was connected to a Masterflex® (Cole-Parmer) pump via platinum silicone pressure-resistant tubing. FIG. 3B is a schematic of the microreactor of the slow flow culture system of FIG. 3A. Stromal cells were seeded into the extra-capillary space via the extra-capillary inlets. Whole BMCs were flowed through the intra-capillary space via the intra-capillary inlets. FIG. 3C is a schematic of the flow of bone marrow cells (BMCs) in the microreactor of FIG. 3B. There was no direct contact between stromal cells and whole BMCs. 0.2 μM pores along the hollow fiber surface allows the bi-directional exchange of secreted factors without allowing cells through. FIG. 3D is a graph of cell counts as a function of fluid flow. All values are means+standard deviation. Data is representative of three biological replicates. The * indicates p-values compared to BM alone. *p<0.05, **p<0.01, and ***p<0.0001.

FIGS. 4A-4B illustrate the results of high- and low-dose 3T3-support cultures. Analysis was performed on whole BMCs at various timepoints after device seeding. Cell counts were normalized to the 1 hour cell count. FIG. 4A is a graph of cell counts of high dose 3T3-support BMCs as compared with BMCs alone over time. FIG. 4B is a graph of cell counts of low dose 3T3-support BMCs as compared with BMCs alone over time. All values are means+standard deviation. Data is representative of three biological replicates. The * indicates p-values compared to BM alone. *p<0.05, **p<0.01, and ***p<0.0001.

FIGS. 5A-5D illustrate the results of 3T3-mediated enrichment cultures. Analysis was performed on whole BMCs at various timepoints after device seeding. FIG. 5A is a graph of the LSK pool as proportion of all live BMCs over time. The cells were enriched with 3T3 support at all timepoints after 1 Hr. FIG. 5B is a graph of the LSK numbers over time. FIG. 5C is a graph of the Lineage^{positive} population over time. FIG. 5D is a graph of the Lineage^{negative} population over time. All values are means+standard deviation. Data is representative of three biological replicates. The * indicates p-values compared to BM alone. *p<0.05, **p<0.01, and ***p<0.0001.

FIGS. 6A-6D illustrate the results of 3T3-mediated enhanced cell cycling cultures. Analysis was performed on whole BMCs pulsed with Carboxyfluorescein succinimidyl ester (CFSE). Cells were sampled at various timepoints for cell cycling properties. FIG. 6A is a graph of a representative gating strategy for LSKs that were within the CFSE^{lo} population. FIG. 6B is a graph of the proportion of LSKs that were CFSE^{lo} over time. FIG. 6C is a graph of the proportion of Lineage^{positive} cells that were CFSE+ over time. FIG. 6D is a graph of the proportion of Lineage^{negative} that were CFSE+ over time. All values are means+standard deviation. Data is representative of three biological replicates. The * indicates p-values compared to BM alone. *p<0.05, **p<0.01, and ***p<0.0001.

FIG. 7 is a graph of an LSK proportion of scatter in 2-D Transwell® (Corning) co-cultures for both high and low stromal doses. All values are means+standard deviation. Data is representative of three biological replicates. The * indicates p-values compared to BM alone. *p<0.05, **p<0.01, and ***p<0.0001.

FIG. 8A is a graph of the raw cell count and viability for a high stromal dose culture in 3T3-seeded micro-reactors. All values are means+standard deviation. Data is representative of three biological replicates. The * indicates p-values compared to BM alone. *p<0.05, **p<0.01, and ***p<0.0001.

FIG. 8B is a graph of the raw cell count and viability for a low stromal dose culture in 3T3-seeded micro-reactors. All values are means+standard deviation. Data is representative of three biological replicates. The * indicates p-values compared to BM alone. *p<0.05, **p<0.01, and ***p<0.0001.

FIG. 9A is a graph of the LSK proportion for a low stromal dose model. All values are means+standard deviation. Data is representative of three biological replicates. The * indicates p-values compared to BM alone. *p<0.05, **p<0.01, and ***p<0.0001.

FIG. 9B is a graph of the LSK cell numbers for a low stromal dose model. All values are means+standard deviation. Data is representative of three biological replicates. The * indicates p-values compared to BM alone. *p<0.05, **p<0.01, and ***p<0.0001.

FIG. 10 is a graph of the proliferation of Peripheral Blood Mononuclear Cells (PBMCs) as modulated in a dose dependent manner by co-culture with Mesenchymal Stromal Cells (MSCs).

FIG. 11 is a graph of the proliferation of PBMCs incubated with MSCs over time.

FIG. 12 is a graph of the proliferation of PBMCs incubated with various cell types.

FIG. 13 is a graph of the proliferation of PBMCs treated with Brefeldin A (BrefA). The x-axis indicates the following culture conditions: Stim (P+)=stimulated PBMCs w/Brefeldin A; Stim (P−)=stimulated PBMCs w/o Brefeldin A; Ctrl (P+)=non-stimulated PBMCs w/Brefeldin A; Ctrl (P−)=non-stimulated PBMCs w/o Brefeldin A; M+P+=stimulated PBMCs in co-culture with MSCs w/Brefeldin A; M+P−=stimulated PBMCs in co-culture with MSCS w/o Brefeldin A.

FIG. 14 is a graph of the proliferation of PBMCs treated with BrefA. The x-axis indicates the following culture conditions: Stim=stimulated PBMCS w/o Brefeldin A; No Stim=non-stimulated PBMCs w/o Brefeldin A+BA=stimulated PBMCs in co-culture with MSCs w/Brefeldin A; -BA=stimulated PBMCs in co-culture with MSCS w/o Brefeldin A.

FIG. 15 is a graph of the proliferation of PBMCs treated with BrefA over time.

FIG. 16 is a graph of the proliferation of PBMCs under a dynamic flow.

FIG. 17 is a graph of the proliferation of PBMCs versus effect of MSC prestimulation on immunosuppressive ability. MSCs were prestimulated with either IFNy, IL-1b, TNFa, TLR3 agonist (Poly I:C), TLR4 agonist (LPS) for either 1 hour or 24 hours. These agents were then washed out and a co-culture with stimulated PBMCs was performed. The X-Axis indicates culture conditions and the y-axis indicates PBMCs proliferation.

FIGS. 18A-18I illustrate a model for proliferation tracking of MSC:PBMC co-cultures. FIG. 18A is a graph illustrating an example of CFSE-based proliferation tracking. Fold change indicates proliferative fold change of a target immune population (PBMC etc.) as compared to maximal proliferative ability. FIG. 18B is a graph of a pharmacodynamic model. FIG. 18C is a chart illustrating a governing equation of the pharmacodynamic model. FIG. 18D is a graph of Fold Change versus MSC:PBMC ratio. FIG. 18E is a graph of Fold Change versus MSCs/well. FIG. 18E is a graph of Fold Change versus MSCs/mL. FIG. 18G is a graph of predictive proliferation versus empirical proliferation for MSC:PBMC ratio. FIG. 18H is a graph of predictive proliferation versus empirical proliferation for MSCs/well. FIG. 18I is a graph of predictive proliferation versus empirical proliferation for MSCs/mL. In FIGS. 18D-18I, teal represents 1.5M PBMS and pink represents 3M PBMC.

FIGS. 19A-19F illustrate flow cytometry data from an MSC:PBMC co-culture experiment. The top row indicates expression of whole proliferative populations. The bottom row indicates expression of each individual proliferative generation. The y-axis is normalized proliferation. FIG. 19A is a graph of CD3 proliferation for various MSC:PBMC ratios. FIG. 19B is a graph of CD3 generations for the various MSC:PBMC ratios. FIG. 19C is s a graph of CD4 proliferation. FIG. 19D is a graph of CD4 generations. FIG. 19E is a graph of CD8 proliferation. FIG. 19F is a graph of CD8 generations. In FIGS. 19A-19F, St=stim, Ct=control, A=1:10, B=1:20, C=1:100, D=1:200, E=1:1000, F=1:2000.

FIGS. 20A-20H illustrate flow cytometry data from an MSC:PBMC co-culture experiment. The top row (including FIGS. 20A, 20C, 20E, and 20G) indicates expression of whole proliferative populations. The bottom row (including FIGS. 20B, 20D, 20E, and 20F) indicates expression of each individual proliferative generation. The x-axis is normalized proliferation and the y-axis is surface marker expression level. FIG. 20A is a graph of CD4 proliferation and CD38 expression for various MSC:PBMC ratios. FIG. 20B is a graph of CD4 proliferation and CD38 expression showing high linearity/correlation. FIG. 20C is a graph of CD4 proliferation and CD25 expression for various MSC:PBMC ratios. FIG. 20D is a graph of CD4 proliferation and CD25 expression showing high linearity/correlation. FIG. 20E is a graph of CD8 proliferation and CD38 expression for various MSC:PBMC ratios. FIG. 20F is a graph of CD8 proliferation and CD38 expression showing high linearity/correlation. FIG. 20G is a graph of CD8 proliferation and CD25 expression for various MSC:PBMC ratios. FIG. 20H is a graph of CD8 proliferation and CD38 expression showing high linearity/correlation. In FIGS. 20A-20F20H, St=stim, Ct=control, A=1:10, B=1:20, C=1:100, D=1:200, E=1:1000, F=1:2000.

FIGS. 21A-21K illustrate multiplex dose response of secreted cytokines. FIG. 21A illustrates the response of IFNa for various MSC:PBMC ratios. FIG. 21B illustrates the response of INFg. FIG. 21C illustrates the response of IL1b. FIG. 21D illustrates the response of IL1ra. FIG. 21E illustrates the response of IL4. FIG. 21F illustrates the response of IL10. FIG. 21G illustrates the response of IL12p40. FIG. 21H illustrates the response of IL17. FIG. 21I illustrates the response of IP10. FIG. 21J illustrates the response of PGE2. FIG. 21K illustrates the response of TNFa. In FIGS. 21A-21K, St=stim, Ct=control, A=1:10, B=1:20, C=1:100, D=1:200, E=1:1000, F=1:2000.

FIG. 22 is a graph of normalized proliferation of PBMC versus time of exposure to MSCs. PBMC proliferation was attained through stimulation with ConA and IL2 for a period of 4 days. MSC Transwell® inserts were removed after 1, 2, and 3 days co-culture initiation to time duration. Proliferation was measured through flow cytometry and CFSE staining.

FIGS. 23A-23K are bar graphs of normalized cytokine secretion versus time of exposure to MSCs. PBMC proliferation was attained through stimulation with ConA and IL2 for a period of 4 days. MSC Transwell inserts were removed after 1, 2, and 3 days co-culture initiation to time duration. Proliferation was measured through flow cytometry and CFSE staining. FIG. 23A illustrates an increase with prolonged MSC exposure for IFNa. FIG. 23B illustrates a decrease with prolonged MSC exposure for INFy. FIG. 23C illustrates no significant change with prolonged MSC exposure for IL1b. FIG. 23D illustrates a slight increase with prolonged MSC exposure for IL1ra. FIG. 23E illustrates a decrease with prolonged MSC exposure for IL4. FIG. 23F illustrates a decrease with prolonged MSC exposure for IL10. FIG. 23G illustrates no significant change with prolonged MSC exposure for IL12p40. FIG. 23H illustrates a decrease with prolonged MSC exposure for IL17. FIG. 23I illustrates no significant change with prolonged MSC exposure for IP10. FIG. 23J illustrates an increase with prolonged MSC exposure for PGE2. FIG. 23K illustrates a decrease with prolonged MSC exposure for TNFa.

FIG. 24 is a graph of normalized proliferation versus culture volume conditions. PBMC proliferation was attained through stimulation with ConA and IL2 for a period of 4 days. Proliferation was measured through flow cytometry and CFSE staining.

FIGS. 25A-25K are bar graphs of normalized cytokine secretion versus culture volume conditions. PBMC proliferation was attained through stimulation with ConA and IL2 for a period of 4 days. Proliferation was measured through flow cytometry and CFSE staining.

FIG. 26 is a graph of normalized proliferation versus Brefeldin A (BrefA) conditions. Either PBMCs or MSCs were treated for 24 hours with Brefeldin A prior to the start of co-culture. PBMC proliferation was attained through stimulation with ConA and IL2 for a period of 4 days. Proliferation was measured through flow cytometry and CFSE staining.

FIGS. 27A-27K are bar graphs of normalized cytokine secretion versus BrefA conditions. Either PBMCs or MSCs were treated for 24 hours with Brefeldin A prior to the start of co-culture. PBMC proliferation was attained through stimulation with ConA and IL2 for a period of 4 days. Proliferation was measured through flow cytometry and CFSE staining.

FIG. 28 is a graph of normalized proliferation versus BrefA conditions. PBMCs or MSCs were treated for 24 hours with Brefeldin A prior to the start of co-culture. PBMC proliferation was attained through stimulation with ConA and IL2 for a period of 4 days. Proliferation was measured through flow cytometry and CFSE staining.

FIGS. 29A-29K are bar graphs of normalized cytokine secretion versus BrefA conditions. Either PBMCs or MSCs were treated for 24 hours with Brefeldin A prior to the start of co-culture. PBMC proliferation was attained through stimulation with ConA and IL2 for a period of 4 days. Proliferation was measured through flow cytometry and CFSE staining.

FIGS. 30A-30D illustrate secreted extracellular vesicle particle size distribution from various bioreactor culture conditions. FIG. 30A illustrates a cell particle count of stimulated PBMC PBMCs versus diameterculture. FIG. 30B illustrates a cell particle count of stimulated PMBCs versus diameterculture. FIG. 30C illustrates a cell particle count of from stimulated PBMC:MSC cultures and MSCs and versus diameter. FIG. 30D illustrates a cell particle count of from stimulated PBMC:MSC cultures and MSCs and versus diameter. NoMSC=stimulated PBMCs; B-M=stimulated PBMCs; PlusMSC=stimulated PBMCs+MSCs; B+M=stimulated PBMCs+MSCs.

FIG. 31 is a graph of the expansion results of PBMCS (lines labeled Stop and Continuous_3) and purified T-Cells (lines labeled Continuous_1 and Continuous_3).

FIGS. 32A-32B are photomicrographs of cells during a stop-flow culture. FIG. 32A shows a pre-flow aggregate of cells. FIG. 32B shows a post-flow aggregate of cells. The scale is 1000 μm.

FIG. 33 is a graph of the expansion results of PBMCs in a stop flow culture using a magnetic pump (line labelled Magnetic) and peristaltic pumps (lines labelled Peristaltic).

FIGS. 34A-34D are schematics illustrating barrier configurations for a co-culture. FIG. 34A illustrates cellular suspensions in both intra- and extra-luminal compartments. FIG. 34B illustrates an encapsulated cell population in an extraluminal space. FIG. 34C illustrates an encapsulated cell population in an intraluminal space. FIG. 34D illustrates a cell-biomaterial gel that is seeded in an extraluminal space.

FIG. 35 is a schematic illustrating a cell culture process.

FIG. 36 is a schematic illustrating an integrated small-scale production/transduction process and quantification of lentiviral engineering using HEK293T cells.

FIG. 37A is a schematic of a transduction strategy in a transwell system

FIG. 37B is a timeline of both the transfection and transduction processes overlapping with the transwell system of FIG. 37A.

FIG. 37C is a schematic of a plate set up for an experiment, n=3 for target cell transduction groups. Experiments were run in duplicate.

FIG. 37D shows GFP expressing HEK293T cells inside of a transwell insert, confirming that transient transfection and, therefore, lentiviral particle production was occurring.

FIG. 37E shows a bottom well containing Jurkat T cells and showing areas of fluorescence confirming transduction of target cells from free floating lentiviral particles.

FIG. 38A illustrates transduction efficiency as determined by flow cytometry when the seeding density of HEK293T cells in the insert was varied.

FIG. 38B illustrates transduction efficiency as determined by flow cytometry when the seeding density of Jurkat T cells in the well was varied.

FIG. 38C shows HEK293T cells that permeated through the transwell into the bottom well due to overcrowding.

FIG. 38C shows transduced Jurkat cells in bottom well of 45% HEK Cell insert.

FIG. 39A1 shows ZEISS flourescent images from 1 μm insert well at 200 μm scale.

FIG. 39A2 shows ZEISS flourescent images from 1 μm insert well at 50 μm scale.

FIG. 39B1 shows ZEISS flourescent images from 8 μm well at 200 μm scale.

FIG. 39B2 shows ZEISS flourescent images from 8 μm well at 50 μm scale.

FIG. 39C illustrates transduction efficiency of Jurkat T cells as determined by flow cytometry results when the porosity of each insert was varied.

FIG. 40 is a schematic illustrating the timeline for a study of PBMC proliferation.

FIG. 41 illustrates MSC:PBMC dose response. The bar graph represents mean+/−SD of 3 samples.

FIG. 41 illustrates MSC/NHDF:PBMC (1:5) dose response. The bar graph represents mean+/−SD of 3 samples.

FIG. 42 illustrates co-culture effect on T-cell proliferation. The bar graphs represent mean+/−SD of 3 samples.

FIG. 43 illustrates the effect of EC phenotypes on T-cell proliferation. The bar graphs represent mean+/−SD of 3 samples.

FIG. 44 illustrates effects of engineered ECs that are activated by shear stress and their proliferative response of PBMC co-culture. The bar graphs represent mean+/−SD of 3 samples.

FIG. 45 illustrates the normalized proliferative response of PBMC co-culture with NHDF (dermal fibroblast), HepG2 (liver), and EA.hy296 (endothelial). The bar graphs represent mean+/−SD of 3 samples.

### DETAILED DESCRIPTION

The use of bioreactors for the ex vivo maintenance and/or engineering of Hematopoietic stem cells (HSCs) and other therapeutic cell types has been applied in various formats; however, such bioreactor forms have presented issues that may prevent a wide scale use, standardized techniques, and/or reproducible results, hindering the ability of such bioreactors to be useful to clinical centers. For example, continuous flow chambers allow for excellent nutrient delivery at the expense of increased, costly media consumption demand (Koller et al., 1993a; Koller et al., 1993b; Palsson et al., 1993; Sandstrom et al., 1996); stirred tanks support larger volumes and allow the monitoring of clonal growth and differentiation though have not maintained HSC phenotypes and stem potential (De Leon et al., 1998; Levee et al., 1994; Sardonini and Wu, 1993; Zandstra et al., 1994); packed bed reactors allow for larger surface area to volume ratios enabling contact of cultured HSCs with growth ligands though are difficult to purify HSCs and have lower recovery yields (Liu et al., 2014; Meissner et al., 1999; Wang et al., 1995). Hollow fiber bioreactors have been used for continuous, recycling flow culture of HSCs, although such bioreactors were not successful in supporting HSC numbers (Sardonini and Wu, 1993; Schmelzer et al., 2015). While these systems have shown benefit, there still remains a need for an integrated system that maintains HSC numbers and phenotype with downstream ease of purification.

One major challenge in ex vivo bioprocessing of HSCs is the loss of stem cell viability and/or phenotype over time in culture. Conventional culture methods rely heavily on media formulations to drive growth while maintaining the appropriate HSC phenotype. Currently, media supplements include various combinations of stem cell factor (SCF), interleukin-(IL) 3, -6, Fms-like tyrosine kinase-3 ligand (Flt3-L), granulocyte colony stimulating factor (G-CSF), fibroblast growth factor-1, -2, Delta-1, and thrombopoietin, which are expensive and have been shown to expand mostly cord blood HSCs ex vivo (Bhatia et al., 1997; Conneally et al., 1997; Delaney et al., 2005; Himburg et al., 2010; Lui et al., 2014; Zhang et al., 2006). To enhance these ex vivo systems, the cellular HSC niche may be partially recapitulated through direct or indirect co-culture with fibroblastic stromal cells (Pan et al., 2017; Perucca et al., 2017) or endothelial cells (Gori et al., 2017). Advanced 3-D culture systems that more readily mimic the spatial organization of stromal and hematopoietic stem and progenitor cells (HSPCs) have shown enhanced long-term engraftment of expanded cells compared to 2-D cultures (Futrega et al., 2017). As such, there is a need for improved bioreactor systems that can be applied to the ex vivo maintenance and/or engineering of HSCs, as well as other therapeutic cell types.

A description of example embodiments follows.

Systems providing for indirect, dynamic co-culturing of two or more cell populations are provided. In one embodiment, the system includes first and second cell populations, separated by a barrier. The barrier physically separates the cell populations from one another while being permeable to the secreted factors of at least one of the cell populations. In further embodiments, three or more cell populations are included. Each cell population may be separated by a barrier from the other cell populations of the system. In yet further embodiments, a single cell population is included in a system, the single cell population separated by a barrier from a compartment configured to contain a second cell population. The first and second cell populations may be different cell types or the same cell types.

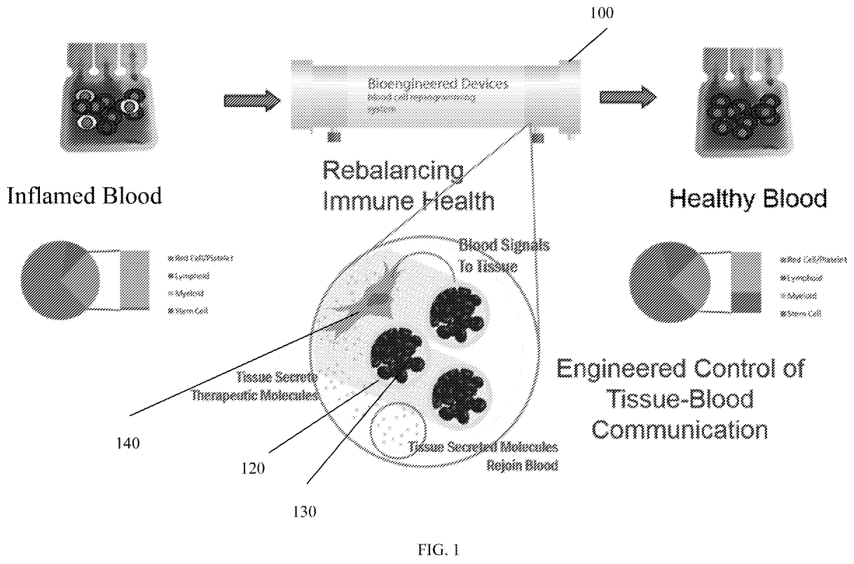

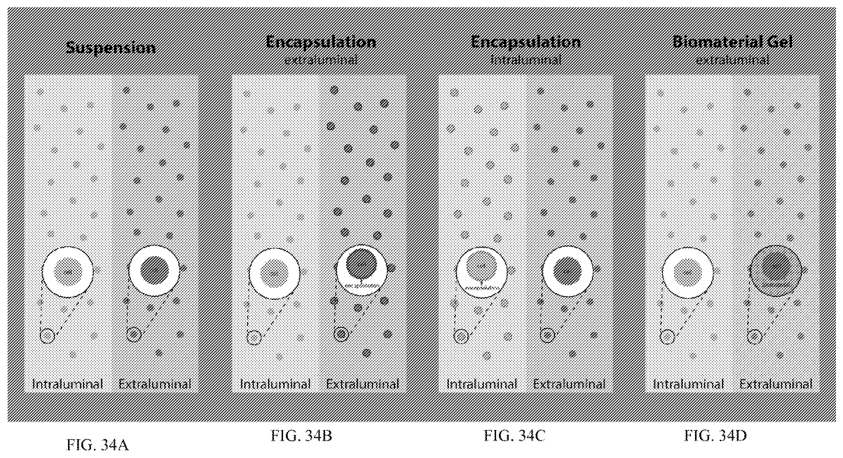

An example of a co-culture system is shown in FIG. 1. In a particular embodiment, the barrier is a semipermeable membrane, such as a hollow-fiber membrane 120. One of the cell populations 130 can be disposed within the intraluminal space of the hollow-fiber membranes, while the other of the cell populations 140 can be disposed in the extraluminal space. The hollow fiber membranes are disposed within a container 100, providing for a closed co-culture system. See also, FIG. 34A. Alternatively, the barrier can be a gel, such as a hydrogel or other biomaterial, with at least one of the first and second cell populations disposed within the gel. See FIG. 34D. In yet another embodiment, the barrier can be a capsule, with at least one of the first and second cell populations disposed within the capsule. See FIGS. 34B and 34C. The encapsulated cell population(s) and/or biomaterials can also be disposed within a container to provide for a closed co-culture system, and, optionally, also separated by a permeable membrane.

The system can further include a fluidic flow driver, which is configured to induce a flow of a liquid suspension of at least one of the first and second cell populations in or through the container. For example, a pump can be included in the system that provides a flow of a cell suspension through the intraluminal space of the hollow-fiber membranes. Alternatively, the fluidic flow driver can be a stirrer configured to induce a flow of a cell suspension disposed in a tank, the suspension including, for example, seeded capsules.

The two cell populations can be stimulator cells and responder cells. Although physically separated, the system facilitates the dynamic and indirect interaction of these two populations through secreted factors. The system can thereby provide for the modification of the responder cells, such as an original hematopoietic cell population, through communication with the stimulator cells. The modified responder cells can be modified to provide for a therapeutic medical product that can be delivered to a patient. The transformed hematopoietic product can be functionally different from the starting, or parental population, such as by having the ability to attenuate disease or a symptom thereof. The reprogrammed cell population can be characterized based on: purity/identity analysis, functional bioactivity, secretory phenotype, gene and DNA expression profiles, surface biomarker expression, as well as other standard characterization techniques.

The physical separation of the stimulator and responder cells advantageously provides for streamlined downstream processing of an end product. Due to the separation of the two cell populations, purification of the responder cells is simplified, obviating the need for additional and unnecessary product handling steps. Systems of the present invention can be located on-site at a clinical institution or off-site at a certified processing and handling facility.

Within a system of the present invention (e.g., a bioreactor system), a stimulator cell type at an effective concentration can communicate with a patient's immune cells ex vivo through a semi-permeable membrane over a period of time. In this way, stimulator cells can continuously and dynamically condition an immune cell therapy without ever having to be injected into the body of the patient. For example, a bioreactor system can be located at a hospital and have an integrated stimulator cell population, such as provided by a cartridge. A bag of hematopoietic cells, obtained from the patient, can then be circulated through the reactor for a specific amount of time to reprogram the hematopoietic cells. The hematopoietic cells can then be formulated and re-administered back to the patient to resolve a disease process.

An example of components of a dynamic co-culture system are shown below in Table 1.

TABLE 1
Components of a dynamic co-culture system

| Component | Specification(s) |
|---|---|
| Fluidic Tubing | Size ID: 0.1-1.0 cm |
| | Size OD: 0.15-2.0 cm |
| | Material: Pharmed BPT, silicone, |
| | PVC, TPE, TPU, Latex, |
| Separating Membrane | Material: polysulfone, polyethersulfone, |
| | cellulose, cellulose aetate, polycarbonate, |
| | polyethylene, polyolefin, polypropylene, |
| | polyvinylidene fluoride |
| | MWCO: 5000 kda − 0.5 um |
| Fluidic Flow Rate | 0-7.7 L/min (volumetric) |
| Fluidic Flow Driver | Peristaltic, syringe, pressure, |
| | centrifugal, magnetic |
| Reservoir | Drip chamber, cell processing bags, |
| | cell culture bags, gas permeable |
| | bags/containers, |
| Bioreactor | Fibers: ≥1 hollow fiber |
| | Directionality: no defined inlet/outlet |
| | Extraluminal side: at least 1 port for |
| | cell introduction |
| | Intraluminal side: at least 2 ports to |
| | allow for flow through |

The system can be dynamic with respect to both cross-talk between cell populations as well as physical movement/agitation of at least one of the cell populations. With regard to cross-talk between the cell populations, the functionality of specific cells types can be enhanced. For instance, inflammatory cytokines secreted by T-cells (IL-1, TNF, IFNy) are known to enhance the immunomodulatory function of mesenchymal stromal cells. The complex milieu of secreted factors is excessively costly to recapitulate with pharmacologic (e.g., chemical and/or protein) agents. The self-renewing properties of cells, through tissue culturing, combined with exposure to the secreted factors of the stimulator cells, provides for economic cell re-programming.

With regard to physical movement/agitation within the co-culture system, dynamic cultures are preferred over static cultures because physical movement increases the diffusion of gases and soluble factors throughout the culture, thereby providing for improved overall cell health, as well increasing shear to break up cell aggregates. Furthermore, dynamic systems are also able to address significantly higher quantities of cells, which facilities the ability to address clinical and commercial scale demands. Non-dynamic systems can become limited by surface area and, if the cells are non-adherent, can result in eventual cell settling; these systems reach terminal confluency much quicker than dynamic systems. Cells can be separated either through permeable membranes (i.e., PES) or permeable substrates (i.e., hydrogel encapsulations). Dynamic system formats can include any one or any combination of the following: hollow fiber membranes, stir flask/tank, microcarriers, rocker (Wave Reactor) bag or flask, roller bottle, packed beads/bed, and tissue engineered constructs.

In some embodiments, a co-culture system is maintained under static conditions for a period of time and the flow of a cell culture medium containing at least one of the responder and stimulator cell populations is discontinuous. For example, static, or substantially static, conditions can be initially maintained upon seeding at least one of the responder or stimulator cell populations to allow the population to establish itself within the system and/or to provide time for secreted factors to perfuse across the barrier and be absorbed by responder cells. Periods of static co-culturing may then follow, interspersed with periods in which the cell culture medium(s) are caused to flow through the system. The flow can be induced, such as by a fluidic flow driver, to break up cell aggregates, which can assist with both cell growth and cell differentiation within the system. The induced flow can be pulsed, such that flow is induced for a defined period of time and/or at defined intervals. For example, a pulsed flow can have a flow duration of at least about 10 seconds, for example, of about 10 seconds to about 30 minutes, of about 10 seconds to about 5 minutes, or of about 10 seconds to about 1 minute. The length of time for which a flow of cell-culture medium is applied can be sufficient to induce shear to break up cell aggregates. The pulses can also be applied at a set frequency. For example, a pulse can be applied at a frequency of about 2 hours to about 40 hours, of about 2 hours to about 6 hours, of about 6 hours to about 12 hours, or of about 12 hours to about 24 hours. For example, T-cells can grow in clonal aggregate. A discontinuous flow configured to break up the aggregates at a defined time or for a defined time period can then permit for the formation of new clusters once flow is halted. Aggregates grown under continuous flow conditions may not form well due to continued shear stress and, therefore, cell numbers can be significantly lower than aggregates grown under discontinuous flow conditions.

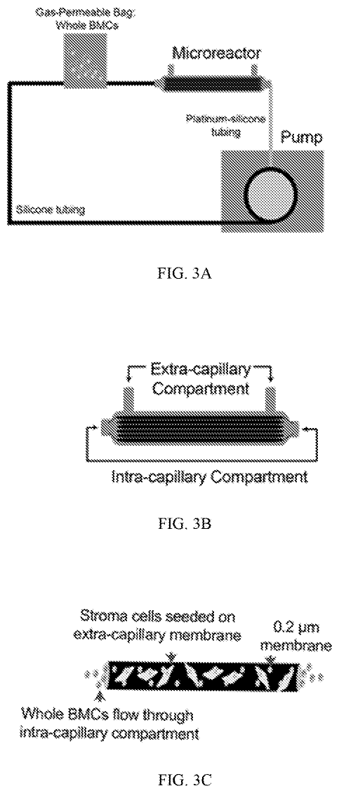

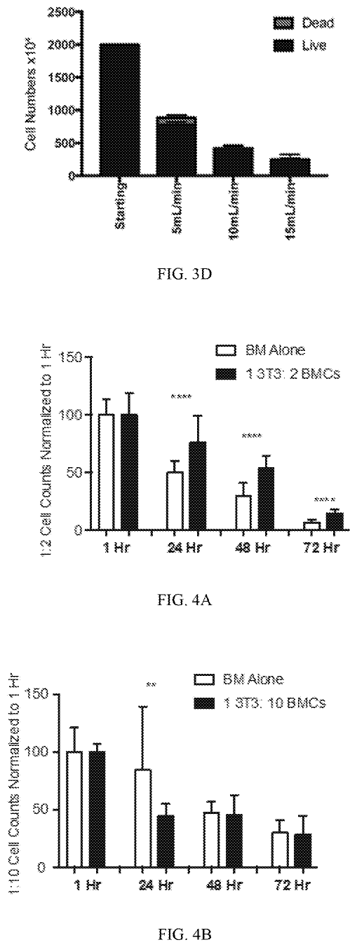

Cartridges can be provided, such as a hollow-fiber membrane capsule (FIG. 3B) seeded with a stimulator cell population. The cartridge may then be inserted into a dynamic co-culture system, such that a patient's blood may be reconditioned with a particular stimulator cell population at a clinical location.

The barrier of the dynamic co-culture system can have a molecular weight cut off (MWCO) of about 30 kDA to about 100,000 kDA, or of about 5000 kDA. Alternatively, the barrier can have a pore size of about 0.00001 μm to about 0.65, or of about 0.5 μm.

Cell conditioning, or cell reprogramming, can occur over at least about 1 hour, at least about 2 hours, or at least about 3 hours. In some embodiments cell conditioning occurs over a period of time of up to about 21 days. In a particular embodiment, cell conditioning occurs over a period of time of about 60 hours to about 120 hours, or over about 96 hours, or over a period greater than about 24 hours. Extended exposure of the responder cells to the stimulator cells can advantageously provide for adequate time for responder cells to reprogram. Reprogrammed cells may be administered to a patient soon after the conditioning protocol ceases. Alternatively, a large batch of cells can be produced, with additional cell “doses” being cryogenically stored for later use.

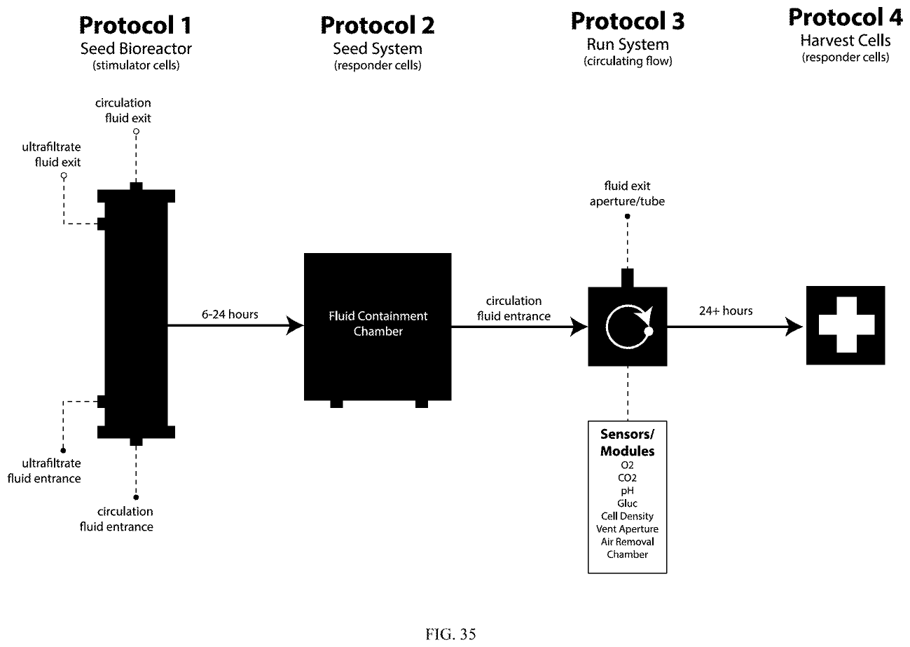

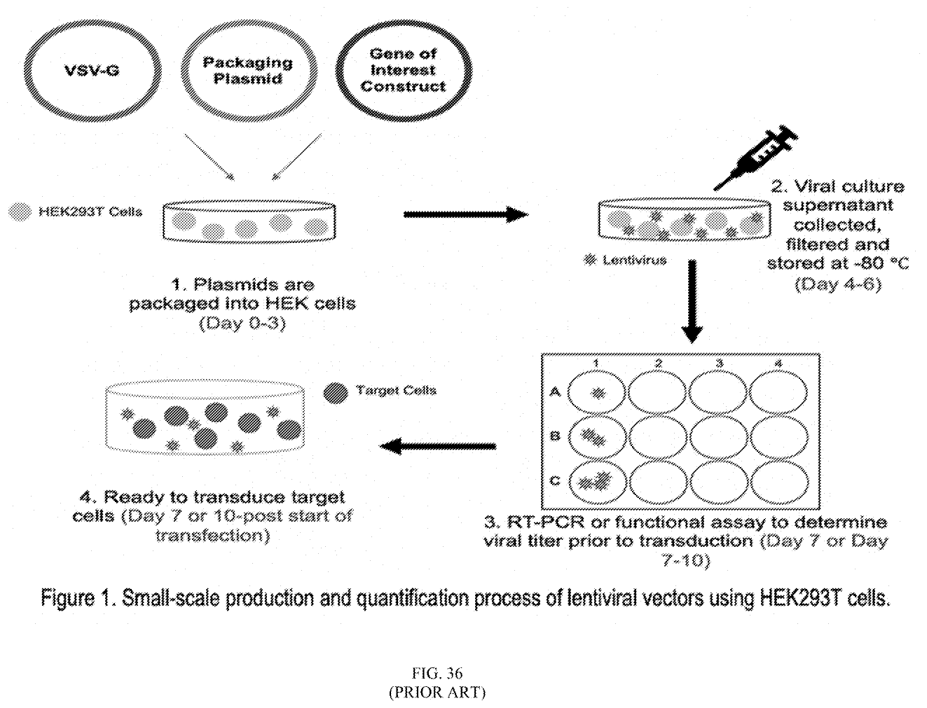

An example of a cell conditioning process, or a process for reprogramming cells, is shown in FIG. 35. In a first protocol, or process stage, a bioreactor (e.g., a container for housing a responder cell population and a stimulator cell population, separated by a barrier) is seeded with stimulator cells. The stimulator cell population can be introduced to the bioreactor by flowing a liquid suspension of the cells into the bioreactor through an ultrafiltrate fluid entrance. The cells may then be incubated for a period of time, such as, for example, about 6 to about 24 hours. The stimulator cell population can be seeded at a density of at least 1 cell/cm², for example, about 1 to about 1,100,000 cells/cm² in the bioreactor. The stimulator cells can further be supplemented with one or more of a growth factor, serum, platelet lysate, and/or an antibiotic.

Following the seeding of the stimulator cells, the responder cells are introduced into the system in a second protocol. In particular, the responder cells may introduced through a circulation fluid entrance and maintained within a fluid containment chamber of the bioreactor. The fluid containment chamber may also serve as a gas exchange tool, particularly if cell culture bags are also used. The cells, including the stimulator and responder cells, can be maintained at about 0.1 to about 21% partial pressure of oxygen during seeding and/or co-culturing.

In a third protocol, the co-culture system is allowed to run for a period of time sufficient for cell reprogramming to occur, for example, about 24 hours or more. During the third protocol, stop-flow processes may occur, where flow of the cell culture medium containing either the stimulator or responder cells, or both, is induced for a period time, followed by periods of substantially static conditions. Several sensing units can be integrated in-line with the flow circuit to allow for real time monitoring of the system and provide for the ability to adjust system parameters (i.e., addition of fresh media). Sensors and modules can include: oxygen, carbon dioxide, glucose, pH, cell density tools, vent apertures, and an air removal chamber.

At the end of the co-culture period, the responder cell population, and, optionally, the stimulator cell population, may then be harvested from the system in a fourth protocol.

One or more pumps can be included in the system to induce a flow of any of the responder cell population, the stimulator cell population, fresh media, and/or reagents to/from the bioreactor. The pumps can be, for example, peristaltic pumps or magnetic pumps (e.g., Levitronix® PuraLev® pumps). As shown in Example 3, stop-flow conditions, particularly as were shown to increase the proliferation of responder cells over continuous co-culturing conditions. As shown in Example 4, flow induced by a magnetic pump was shown to also increase the proliferation of responder cells, as compared with flow induced by peristaltic pumps.

The bioreactor, or tubing extending to/from the bioreactor, can further include an air removal chamber and/or vents to provide for the expulsion of gases from the otherwise closed system. An air removal chamber and/or vents can also be provided to remove or trap bubbles within the system that are harmful to cells.

Stimulator cells included in a dynamic co-culture system can be stromal cells, viral packaging cells, antigen exposed cells, young blood cells, microbial cells, endothelial cells, fat cells, fibroblasts, cancer cells, and/or neurons. The stimulator cells can be disposed within a tissue.

Responder cells can be immune cells, bone marrow cells, and/or red blood cells. In a particular embodiment, the responder cells are hematopoietic stem cells or hematopoietic progenitor cells. In another particular embodiment, the responder cells are leukocytes.

In another embodiment, the stimulator cells are mesenchymal stem cells and the responder cells are T-cells.

The secreted factors to which the responder cells are exposed can be nucleic acids, such as mRNA, microRNA, circular RNA (circRNA), and DNA. Alternatively the secreted factors can be growth factors, chemokines, and/or cytokines. The secreted factors can be included in exosomes of the stimulator cells, which can then be endocytosed by the responder cells.

Examples of stimulator and responder cell pairings are shown in Table 2. Leukocytes may be considered a type of immune cell and are included within the immune cell categories reflected in Table 2. Young cells include cells taken from a subject during embryonic, fetal, or early adolescence stages, for example, up to about age 18 for a human subject. Fat cells include white, brown, and beige adipose cells.

TABLE 2
Stimulator and Responder Cell Pairings

| Stimulator | Responder | Target Mechanism | Indications |
|---|---|---|---|
| Stromal Cell | Immune Cells | Immunomodulation | Autoimmunity, |
| | | | Transplant Rejection, |
| | | | Allergy, Inflammation |
| Stromal Cell | Immune Cells | Immune Cell | CAR-T, cytotoxic T-cells, |
| | | Proliferation | clinical/commercial |
| | | | scale cell production |
| Packaging Cell | Bone Marrow | Genetic | Gene Therapy |
| | Cells | Enginering/Editing | (eg CAR-T cells, orphan diseases) |
| | | (DNA, RNA, derivatives thereof) | |
| Antigen-Exposed | Immune Cells | Vaccination | Tolerance, Immune |
| Cell | | | Stimulation to |
| | | | Infection Disease |
| | | | and Cancers |
| Young Cells | Immune Cells | Regeneration | Hematopoietic Cell |
| | | | Boosting and |
| | | | Differentiation, Aging |
| Microbial Cell | Immune Cells | Microbiome-Related | GI Diseases, Cancer, |
| | | | Metabolism, Allergy, |
| Endothelial Cell | Immune Cells, | Metabolic Disorders | Diabetes, Heart |
| | RBCs | | Disease, Liver |
| | | | Diseases |
| Young/Fat Cells | Immune Cells | Neurodegenerative | Alzheimer's, |
| | | Disease | ALS, Parkinson's |
| Neuron | Immune Cells | Immunomodulation | Autoimmunity, |
| | | | Transplant Rejection, |
| | | | Allergy, Inflammation |

In another embodiment, a composition is provided having a population of reprogrammed cells. The reprogrammed cells include biomolecules (e.g., nucleic acids, proteins) originating from a different cell population (e.g., stimulator cells). The reprogrammed cells can exhibit one or more additional or modified functional activities than their parental population. As understood herein, a “parental population” is a cell population that has not been exposed to the secreted factors of the different cell population. The term “reprogrammed cells” refers to a population of the parental cells that have been exposed to the secreted factors of the different cell population. The reprogrammed cells may additionally express cell surface markers not expressed by the parental population. The reprogrammed cells can have an additional or modified activity compared to the parental cell population, such as a modified T-cell proliferation activity in vitro.

The composition can be formulated in an acceptable vehicle for administration to a subject with an immunological disease. A method can include administering the composition to a patient in need thereof. For example, the composition may be provided according to a therapeutic regimen for increasing or decreasing production of one or more anti-inflammatory cells or to treat a disease, disorder, or condition associated with inflammation. Such diseases, disorders, and conditions include, for example, rheumatoid arthritis, type I and type II diabetes, ankylosing spondylitis, amylotrophic lateral sclerosis, scleroderma, Bechets disease, hemophagocytic lymphohistiocytosis (HLH) and macrophage activation syndrome (MAS), ulcerative colitis, Crohn's disease, celiac disease, multiple sclerosis, myocardial infarction, neoplasm, chronic infectious disease, systemic lupus erythematosus, bone marrow failure, acute kidney injury, sepsis, multiple organ dysfunction syndrome, acute liver failure, chronic liver failure, chronic kidney failure, pancreatitis, and Grave's disease.

Examples of bioreactor systems are described in Examples 1 and 2 herein. In particular, hematopoietic stem and progenitor cells (HSPCs) were stabilized and enriched by an indirect fibroblast feeder coculture (Example 1). Also, data demonstrating the reprogramming of immune cells using Mesenchymal Stromal Cells (MSCs) was obtained (Example 2).

A theorized mechanism of action for the reprogramming of immune cells are exosomes secreted by MSCs. Exosomes contain genetic material, such as RNA, from their host cells, which, in Example 2, are MSCs. Exosomes can subsequently be endocytosed by recipient cells, which, in Example 2, are T-Cells. The −Cells may undergo a conformational change by adopting and integrating MSC-derived material. A clear shift in measured exosomes was observed in MSC:PBMC cultured compared to controls. See also, Example 5. This initial data demonstrates that MSCs do indeed release detectable exosomes in the bioreactor system.

### EXEMPLIFICATION

### Example 1. Hollow-Fiber Bioreactor System

A scalable hollow-fiber bioreactor system was created in which a mouse embryonic fibroblast cell line, known to provide hematopoietic support (Roberts et al., 1987), was indirectly co-cultured as a feeder layer with mouse HSCs in a continuous, concentrated, and recycling flow to stabilize and enrich HSCs for short-term bioprocessing. Since feeder cells were separated from HSCs by the hollow fiber membrane in the present system and subject to circulatory flow, the isolation of HSCs was simplified. Proof-of-concept studies were conducted to screen appropriate purified stromal feeder cells, develop a coculture method in a microreactor system, and evaluation of the indirect stabilization and enrichment of mouse bone marrow HSCs.

Animals and Bone Marrow Cell Suspension

Primary murine whole BM cells were isolated from 6-8 week old female C57 BL/6 mice femurs and tibia by flushing the central marrow using a 28-gauge needle and 3 mL syringe. Flushed marrow was triturated into a single cell suspension with a 22-gauge needle and 3 mL syringe and then subsequently filtered through a 70 μM nylon mesh to exclude tissue debris from the cell preparation. Cell were washed with culture media and then subjected to ammonium-chloride-potassium (ACK) lysis buffer (Biolegend, CA, USA) treatment for the removal of red blood cells. BM cells were then counted for experimental use using the cell viability dye, Trypan Blue (ThermoFisher Scientific, USA) and hemocytometer. Culture media was made with RPMI (Roswell Park Memorial Institute Medium; Gibco, USA) supplemented with 10% FBS, 1% penicillin/streptomycin, 10 mM HEPES (ThermoFisher Scientific, USA), and 1 mM Sodium Pyruvate (ThermoFisher Scientific, USA).

2-D Transwell Co-Culture

Initial optimization experiments were conducted in 2-D transwell co-cultures to allow a higher degree of throughput and parameter assessment to determine the optimal culture criteria: stromal cell type and dose. Corning 24-well plate transwells containing 0.4 μM pores (Grenier Biosciences, Austria) were used for these cultures. Stromal cells were seeded with either 2E5 (low stromal dose: 1:10) or 4E5 (high stromal cell dose: 1:2) cells, 24 hours prior to whole bone marrow cell (BMC) addition. BMCs were seeded at either 2E6 or 8E5 cells respectively, and then the co-culture was incubated for 72 hours at 37° C. Cells were enumerated and analyzed by flow cytometry for their HSPC population. Cells were cultured with standard RPMI cell culture media as per description above.

3-D Micro-Reactor Setup

The hollow fiber micro-reactor was purchased from Spectrum Labs (CA, USA). The intra- and extra-capillary spaces of the micro-reactor hollow fibers were filled with sterile-filtered 0.5M sodium hydroxide (NaOH) and left for 1 to 2 hours at 37° C. for reactor sterilization. The NaOH was flushed out multiple times with sterile phosphate buffered saline (PBS) (Sigma, USA). All fluidic connectors and tubing (Mastedlex PharMed® BPT Tubing and Platinum-cured Silicone Tubing, IL, USA) were similarly soaked and flushed with 0.5M NaOH for at least one hour, and washed with sterile-filtered PBS. FIG. 3A illustrates the setup of the device for co-culture.

3-D Micro-Reactor Seeding and Sampling

Micro-reactors were seeded with either 7E6 (high stromal dose: 1:2) or 3 to 4E6 (low stromal dose: 1:10) 3T3 stromal cells 24 hours before co-culture was initiated. At 24 hours, seeded micro-reactors were gently washed with culture media, attached to cell culture bags (Origen) containing either 14E6 (high stromal dose: 1:2) or 30 to 40E6 (low stromal dose: 1:10) whole BMCs respectively. Flow was introduced by using a Masterflex (IL, USA) peristaltic pump which was run at 4 mL/min. 250-500 μL sample aliquots were taken at 1, 24, and 48 hour timepoints for cell quantification and flow cytometric analyses. Co-cultures were harvested at 72 hours after administering flow and analyzed similarly.

Stromal Cell Cultures

Murine mesenchymal stem cells were purchased from Gibco. Cells were subcultured in media composed of sterile a-MEM (Gibco, MA) supplemented with 10% heat inactivated fetal bovine serum (FBS; Atlanta Biologicals, GA), 1% penicillin/streptomycin (Gibco, MA), 1% antibiotic-antimycotic (Gibco, MA) and used for experiments within 1-3 passages from the initial vendor stock. 3T3 cells were purchased from the ATCC (American Type Culture Collection) and expanded as per their recommended instructions; in DMEM supplemented with 10% FBS (Atlanta Biologicals, GA) and 1% penicillin/streptomycin (Gibco, MA).

Flow Cytometric Analyses

Cells were stained for 20-30 minutes at 4° C. in the dark with directly labeled human monoclonal antibodies directed against CD11b-, CD11c-, Gr1-, CD3-, CD4-, CD8a-, CD19-, B220-, NK1.1, and TER119-conjugated to biotin, cKit (CD117)-APC, Sca1-PECy7, and streptavidin APC Cy7 (BD Biosciences and eBiosciences, USA). Cells were washed and then resuspended with PBS supplemented with 2% FBS and 2 mM Ethylenediaminetetraacetic acid (EDTA; Gibco, USA). Flow cytometric analysis was performed using FACS LSRII (BD Biosciences, USA) and FlowJo Software (USA).

Cell cycling was assessed with carboxyfluorescein (CFSE) incorporation at the start of the co-culture. FO peaks were determined using the control sample of BMCs flowed through the micro-reactor without stromal support.

Statistical Analyses

All statistical tests were performed using GraphPad Prism. Differences between groups were tested using Student's t-test whereby a p-value of <0.05 were considered to be statistically significant. All comparisons were performed relative to BM alone micro-reactor co-cultures. All experiments were repeated with at least three biological replicates.

Results: Murine Embryonic Fibroblast Cell Line Supports LSK Enrichment in 2-D Non-Contact Dependent Co-Cultures.

Stromal cells have previously been shown to enhance hematopoietic support of whole BMCs in vitro. In the present study, two criteria for selecting an optimal stromal cell type were applied: 1) the ease of cell isolation, maintenance, and expansion for “off-the-shelf” use as well as 2) the hematopoietic supporting potential of HSPCs. To determine which stromal cell type would be utilized for supporting whole BM cells in vitro, a comparison of HSPC supporting capacity of bone marrow mesenchymal stromal cells (MSCs) to 3T3 mouse embryonic skin fibroblast cell line was performed. As a control, a non-stroma containing group was included. To screen for optimal co-culture conditions, a 2-D non-contact dependent Transwell setup was adapted that mimics indirect co-culture with higher throughput experimental advantage.

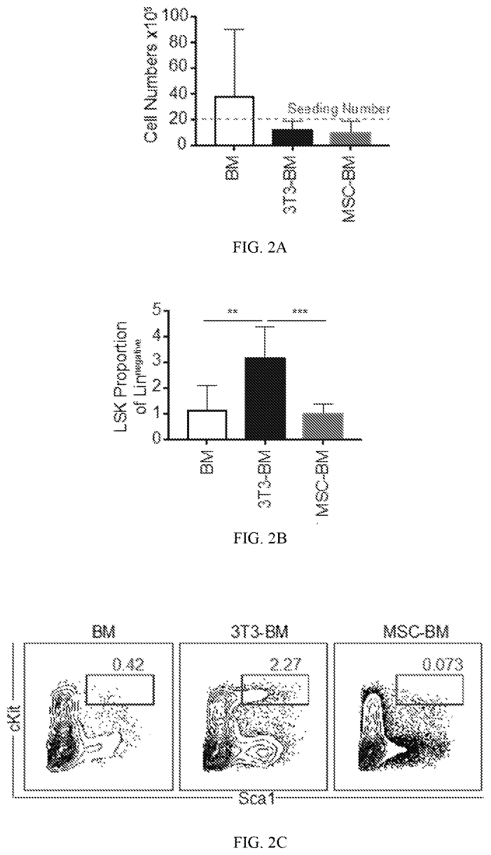

At 72 hours after co-culture with and without stromal support, the whole BMC compartment was enumerated. Stromal support maintained the total number of whole BMCs (FIG. 2A) at their initial seeding numbers of 2×10⁶ input cells over 3 days. Subpopulations of BMCs were then analyzed into the more therapeutically relevant LSK population, phenotypically defined as being Lineage^{negative} Sca^{positive} cKit^{positive} (LSK) (FIG. 2B). Interestingly, the 3T3 fibroblast cell line exhibited a superior capacity to enrich for the LSK population, both within the Lineage^{negative} and whole BMC pools (FIGS. 2B-C and FIG. 7). There were no phenotypic anomalies in the LSK profiles of 3T3 stromal-supported BMCs (FIG. 2C). These LSK enrichment findings were observed in a range of 1:2 and 1:10 of 3T3 cells to whole BMCs (FIG. 7) suggesting a dynamic working range with which to scale up. The results indicate a previously undiscovered capacity for 3T3 fibroblasts to support the enrichment of mouse HSPCs from whole BM. The “off-the-shelf” properties and superior-hematopoietic supporting potential led to a scale up with 3T3s for all subsequent experiments in our hollow fiber microreactor.

Results: Whole Bone Marrow Inoculation and Stability in a Hollow Fiber Microreactor.

FIGS. 3A-C schematically illustrate the hollow-fiber bioreactor system. Bioreactors are traditionally employed for the scale-up of a single cell type. A hollow-fiber system was used for streamlined exchange and replenishment of fresh media to support large scale cell expansion. This system was modified as a co-culture tool wherein stromal cells were seeded in the extraluminal space while BMCs were flowed through the interior fiber lumen to investigate the potential of an engineered tissue layer to support LSKs during continuous suspension culture (FIGS. 3A-C).

The micro-reactor setup consisted of cells flowing downward through specialized tubing from a gas-exchange bag through a Masterflex pump, which drove the cells through hollow fibers via their intra-capillary inlet (FIGS. 3A-B). The extra-capillary surface was seeded with stromal cells through the marked inlets (FIG. 3B). The hollow fiber membranes were composed of hydrophillic polyethersulfone (PES) and each fiber has a molecular weight cut off of 0.2 μM pores that allow the bidirectional exchange of only acellular fluid (FIG. 3C). PES was selected for its highly durable nature in withstanding high temperatures and constant fluid exposure. Masterflex PharMed® BPT Tubing was used to allow the flow of BMCs from the bag to the micro-reactor by looping through the Masterflex pump head (FIG. 3A). This PharMed BPT tubing is made from platinum silicone and was used because it demonstrates low levels of spallation, high resistance to acids and alkalis, and can withstand high pressures with minimal cell shearing. Hematopoietic cells at a density of 14×10⁶ cells were tested for viability while circulating in the intracapillary compartment under different flow rates (5 mL/min, 10 mL/min, and 15 mL/min). A total cell count drop after whole BMCs were subjected to 1 hour of flow due to the attachment of monocytic cells and then remained relatively unaltered for the 72 hour culture period. Higher cell counts were observed at lower flow rates after the 72 hour period, so the lowest flow rate on the Masterflex pump of 4 mL/min was selected (FIG. 3D).

Results: Stromal Cell Support Rescues Cell Loss in Hollow Fiber Bioreactor.

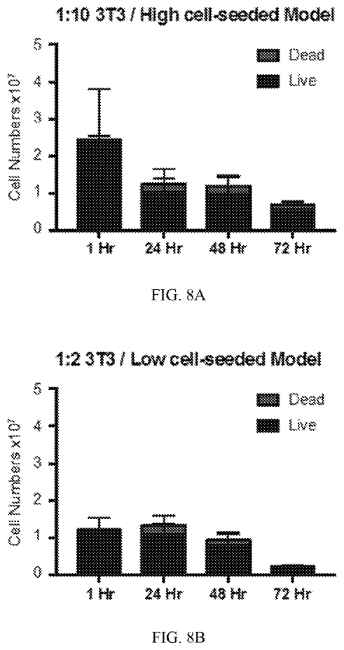

Static co-cultures indicated that 3T3 fibroblasts exhibited superior hematopoietic support over MSCs and un-supported BMCs (see FIG. 2). To verify this result in the micro-reactor system, freshly-thawed 3T3s were seeded on the extraluminal surface of the hollow fibers 24 hours prior to loading the device with primary BMCs to allow 3T3s time to attach and have stable function. 3T3-containing co-cultures offered a statistically significant protective advantage over BMCs when compared to non-stromal supported marrow (FIG. 4A). This was consistently seen until the 72 hour timepoint. A high dose of stromal support was required of 1 3T3 cell to 2 BMCs in order to maintain LSK numbers (FIGS. 4A-B). There were no observable differences in the numbers of dead cells detected over time suggesting that circulating cells likely adhered to system components and were unaccounted for in the suspension cell counts (FIGS. 8A-8B).

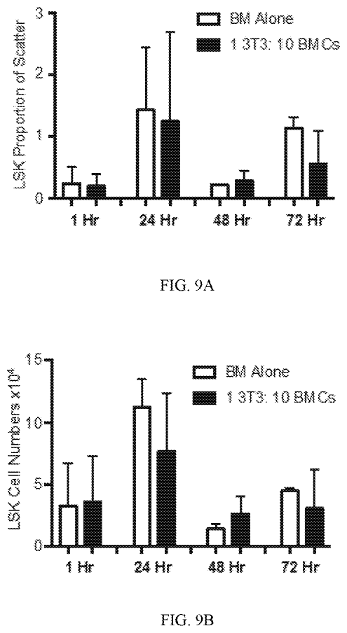

Similar to that seen with counts normalized to the 1 hour samples, raw counts at the 1, 24, 48, and 72 hour timepoints also indicated that 3T3 fibroblasts at a dose of 1 stromal cell to 2 BMCs were able to prevent the trending decline in cell number seen with non-supported BMCs (FIGS. 9A-B). Following this result, all subsequent experiments to dissect the hematopoietic compartment of the BMCs from the micro-reactor were conducted with the 1:2 high stromal dose model.

Results: 3T3 Fibroblasts Enrich for LSKs.

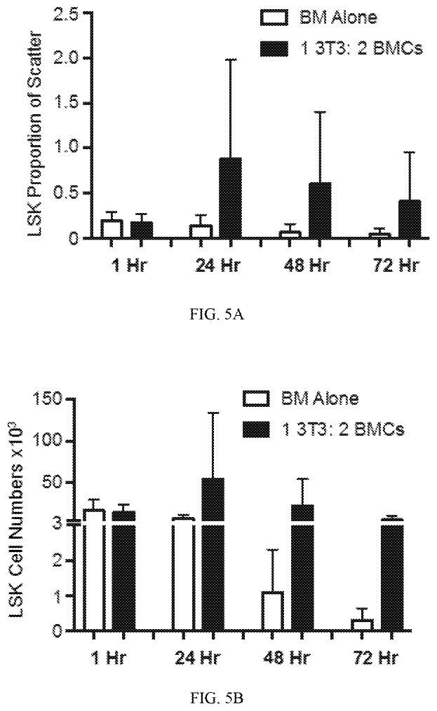

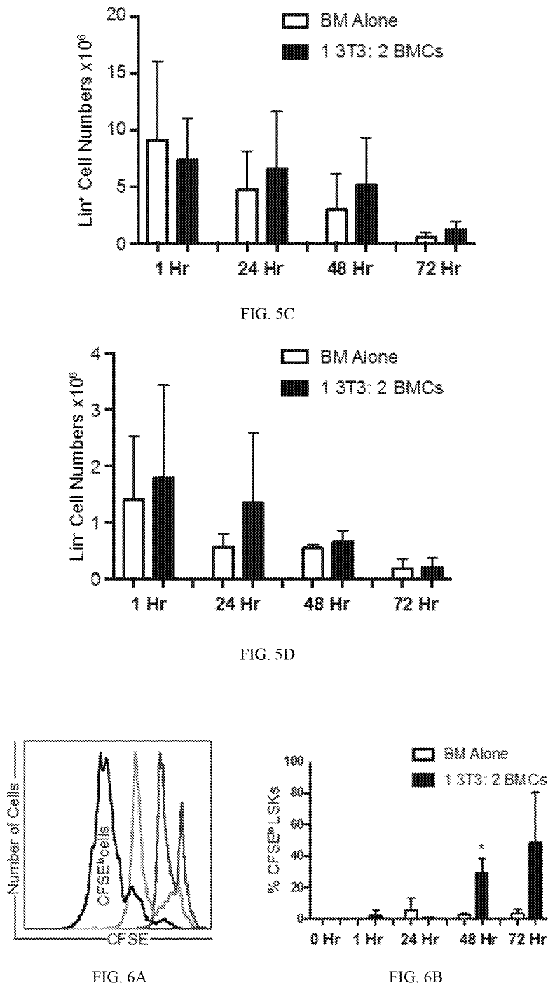

The ability of the 3T3-seeded micro-reactor to stabilize cell counts for 48 hours forms a strong foundation for its exploration to expand BM-derived cells; however a key therapeutic interest within this pool of cells is the hematopoietic stem and progenitor compartment. To evaluate this HSPC population, the sampling timepoints were analyzed by flow cytometry for the LSK phenotype (FIGS. 5A-B). The results indicate a very strong trend towards the enrichment of LSK cells within the whole BMC population from the 3T3-seeded micro-reactors (FIG. 5A). The LSK numbers were expanded in a similar fashion with 3T3 support when compared to the non-stromal supported marrow (FIG. 5B). A similar analysis was also performed with the low stromal dose device (FIGS. 9A-9B). This LSK-enriching potential was only seen with the higher dose of 1 3T3 cell to 2 BMCs (FIGS. 9A-9B and FIGS. 5A-B).

The stromal populations within the pool of whole BMCs were then evaluated: Lineage^{positive} and Lineage^{negative} cells. Interestingly, there were no detectable changes in the numbers of Lineage^{positive} and Lineage^{negative} hematopoietic-supporting cells within the whole BMC population (FIGS. 5C-D).

Results: LSKs Exhibit Enhanced Intrinsic Cell Cycling with 3T3 Support.

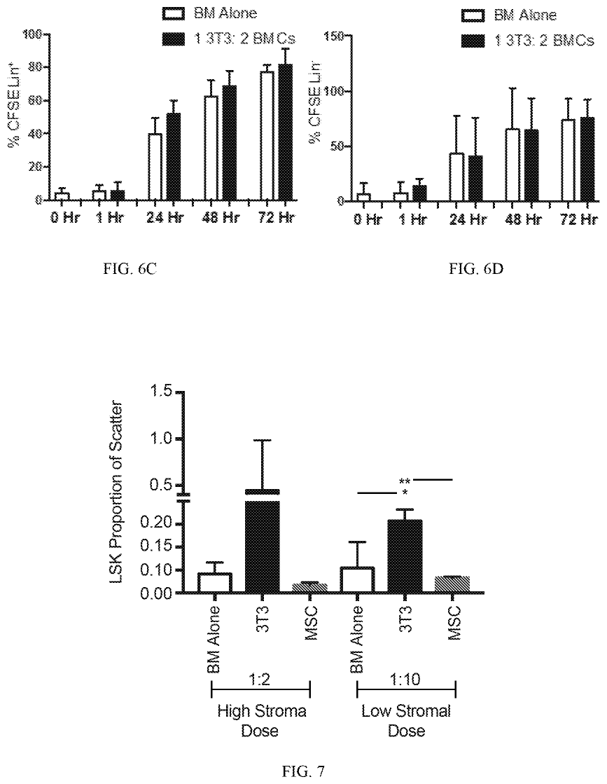

Since no differences were detected within the stromal compartments of the whole BMCs that were used to flow through the 3T3-seeded micro-reactors, it was hypothesized that the observed enrichment of the LSK population was a result of intrinsic changes. Therefore, the whole BMCs were pulsed with CFSE prior to micro-reactor loading to investigate the intrinsic cell cycling patterns associated with 3T3 support. FIG. 6A illustrates the CFS^{lo} pool of LSKs analyzed. As expected, few LSKs were cycling at the 0 to 1 hour timepoints (FIG. 6B). At the later timepoints of 48 and 72 hours, there were significantly (only 48 hour timepoint was statistically significant) larger pools of LSKs that were CFSE^{lo}, when 3T3 were supporting BMCs (FIG. 6B).

To assess whether this was merely a result of all BM cells expanding within the 3T3-seeded reactors, the cycling properties of the stromal Lineage^{positive} and Lineage^{negative} cells were further analyzed (FIGS. 6C-D). While there was a gradual increase of CFSE incorporation in both these cell types with increasing time in the micro-reactor, there were no differences between BMCs with and without stromal support (FIGS. 6C-D).

### Example 2. Examples of Reprogramming Immune Cells Using Mesenchymal Stromal Cells

A series of experiments were performed to show that Mesenchymal Stromal Cells (MSCs) can inactivate T cells by indirect coculture, thereby leading to a new anti-inflammatory cell composition. The dose of MSCs, timing of coculture, the volume of coculture, and phenotypic changes that occur to T cells were reduced to practice in an in vitro system. The in vitro system included a standard tissue culture multi-well plate in combination with Transwell inserts. T-cells were placed in the well bottoms while MSCs were placed in the Transwell inserts. The Transwell insert allows for the transport of secreted factors between the two cell populations but eliminates and possibility of direct cell contact and interaction.

MSCs are known to depress CD3+ T-Cell proliferation of activated T-Cells (mitogens, CD3/CD28) when co-cultured with MSCs (direct or indirect through Transwell inserts). This can further be observed in generational differences, whereby a clear reduction in proliferative generations are detectable with T-Cells cultured in the presence of MSCs. This function was demonstrated to have a dose dependent function. T-Cell activation markers also showed good correlation with proliferation levels. MSCs were able to depress CD38 and CD25 (intermediate and late markers) in co-culture conditions in a dose dependent manner. In relation to T-Cell activation and proliferation, MSCs also altered T-Cell secretome under stimulation. A dose dependent decrease in pro-inflammatory cytokines (TNFa, IL1b, IL17) during indirect co-culture was observed.

Dose Response & Volume

FIG. 10 illustrates the dose response and volume of MSC:PMBC interactions. MSC/PBMC interaction can be directly modulated in a dose dependent manner. Changes in culture volume were shown to significantly impact proliferative outcomes. The Groups of FIG. 10A are as follows: Group A: 1.5M PBMCs, 0.8 mL; Group B: 1.5M PBMCs, 1.6 mL; Group C: 3.0M PBMCs, 0.8 mL; Group D: 3.0M PBMCs, 1.6 mL; Group E: 0.75M PBMCS, 0.8 mL.

MSC: PBMC Timing

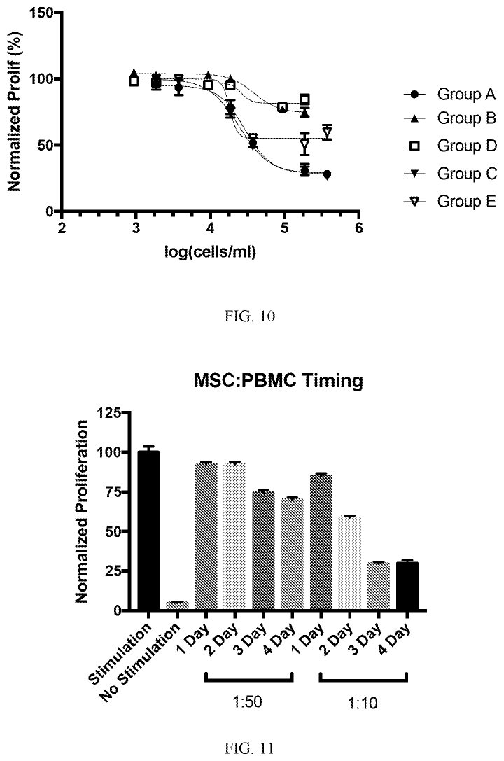

The duration of MSC presence was shown to significantly effect PBMC proliferation, as shown in FIG. 11.

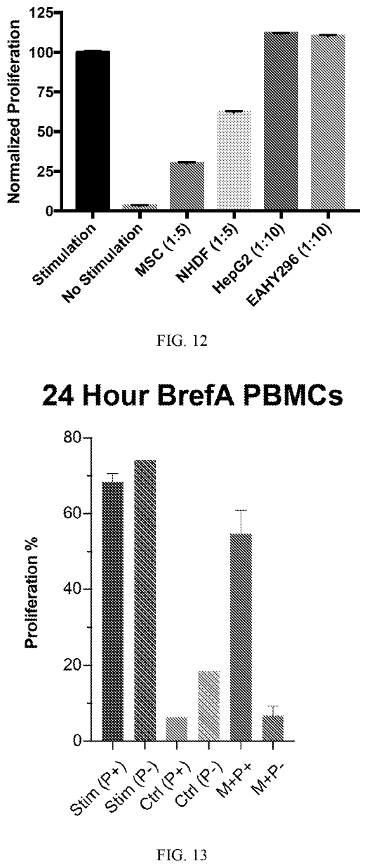

Additionally, the immunoproliferative effects were enhanced, but not specific to MSCs. There was also apparent enhanced proliferation through incubation with other cell types, as shown in FIG. 12.

24 Hour BrefA PBMCs

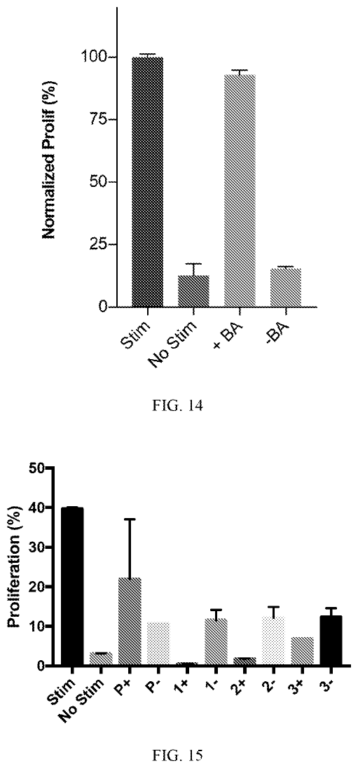

Brefeldin A treatment of PBMCs was shown to reduce the efficacy of MSCs, which indicates a cross-talk mechanism. A 24 hour BrefA treatment was performed on PBMCs, the results of which are shown in FIG. 13. Additionally, Brefeldin A treatment of MSCs was shown to reduce the efficacy of MSCs, supporting the therapeutic nature of secreted factors. A 24 hour BrefA treatment was performed on MSCs, the results of which are shown in FIG. 14.

Brefeldin A treated MSCs demonstrated enhanced function over controls. MSCs were treated for 3 hours prior to co-culture and on Day 1, 2, 3, the results of which are shown in FIG. 15.

MSC: PBMC Bioreators

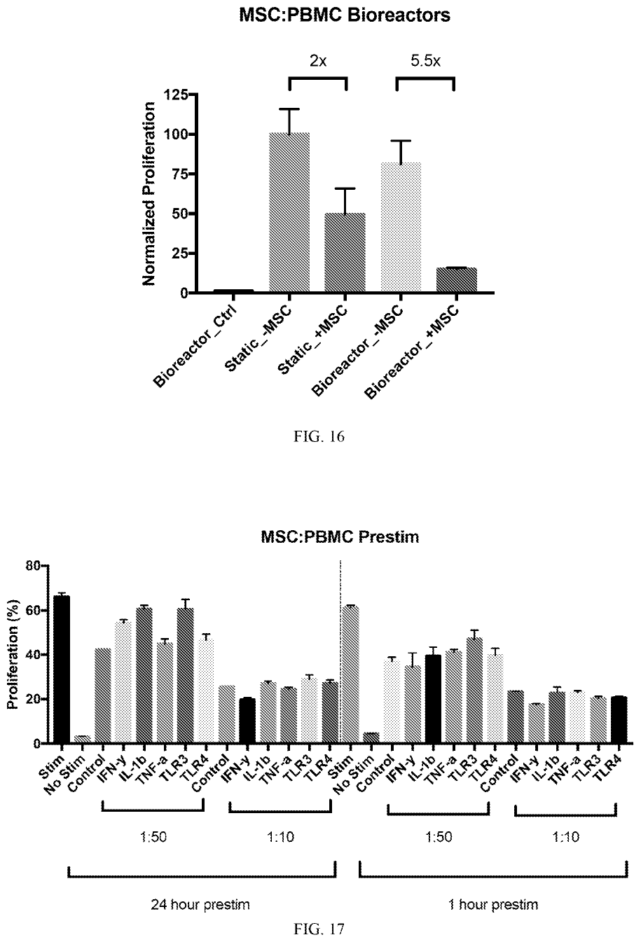

In a dynamic culture, enhanced immunosuppressive effects under dynamic flow of PBMCs were observed. The co-culture was performed after 24 hour PBMC stimulation, demonstrating the ability of MSCs to rescue an inflammatory insult. The results are shown in FIG. 16.

MSC: PBMC Prestimulation

The licensing of MSCs with inflammatory cues was shown to not significantly enhance function. The results are shown in FIG. 17.

IFN-γ appears to be the only cytokine with good effect at 1:10. At 1:50, the dominating factor was shown to be the number of cells that cannot be overcome by licensing.

Proliferation Tracking

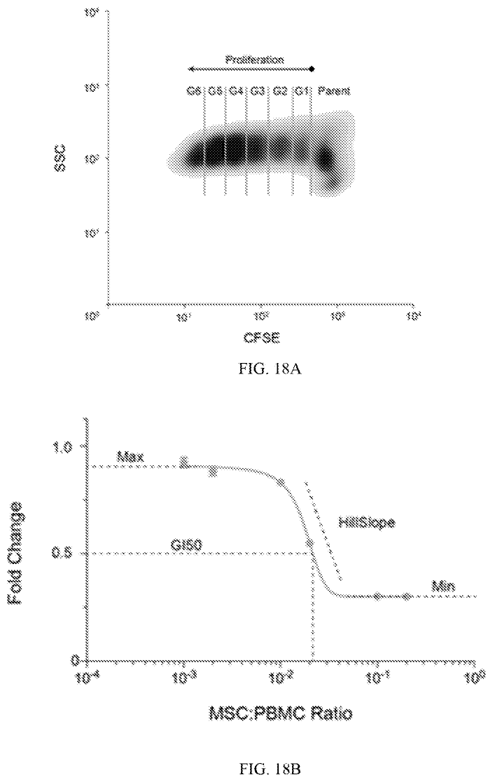

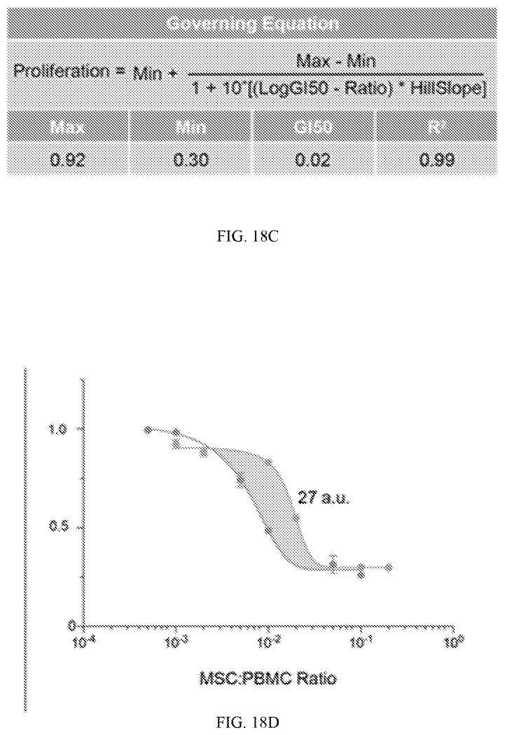

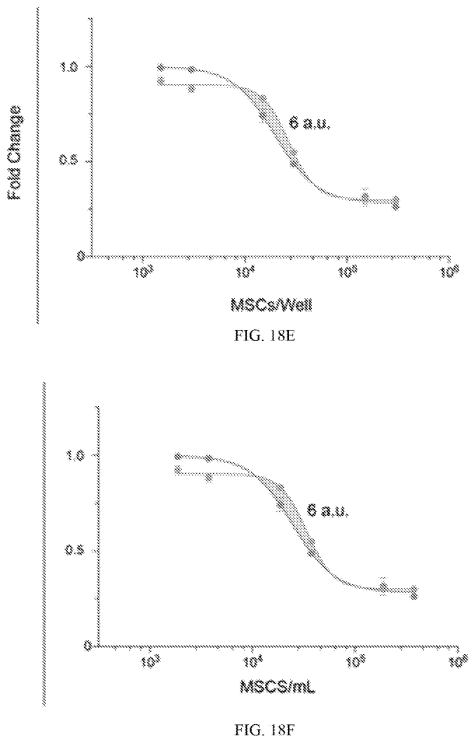

Proliferation tracking was performed using a standard CFSE dye. Prior to co-culture initiations, PBMC populations were stained with this dye. Upon cellular division, this dye becomes divided between parent and daughter populations thus resulting in overall signal reduction. This is readily seen in the leftward shift in signal intensity as the generation increased (FIG. 18A). This can be easily detected through standard flow cytometry and allows for the detection of individual generations.

An example of a pharmacodynamic model is shown in FIGS. 18A-18I. FIG. 18A illustrates an example of CFSE-based proliferation tracking. The pharmacodynamic model and governing equation is shown in FIGS. 18B-18C. Modeling results are shown in FIGS. 18D-F, with the shaded area in arbitrary units (a.u.). The predictive ability of the model is shown in FIGS. 18G-I.

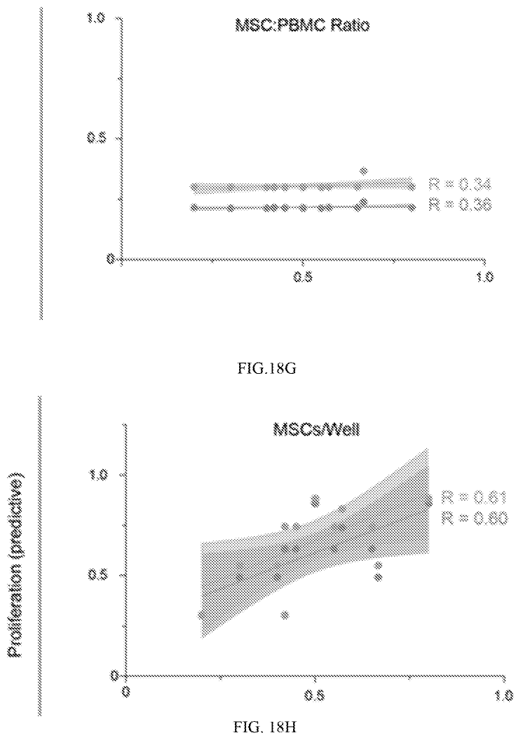

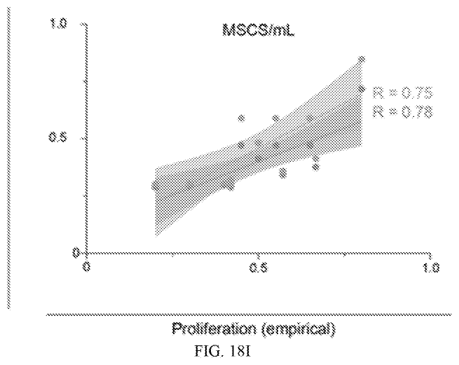

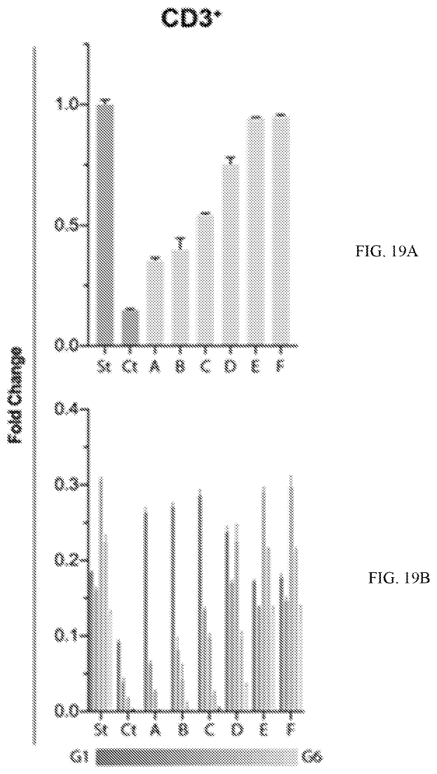

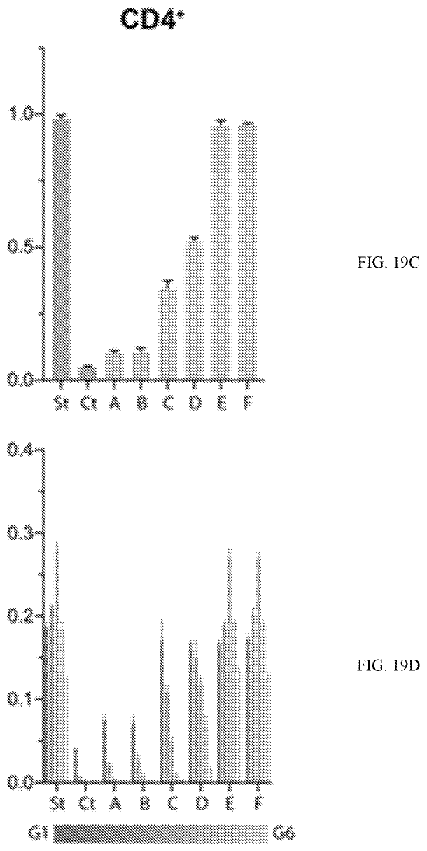

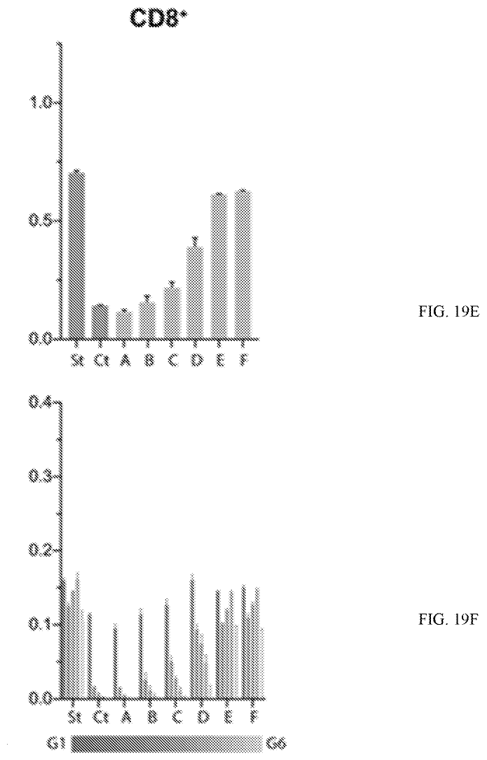

FIGS. 19A-19F illustrate flow cytometry data from an MSC:PBMC co-culture experiment. The top row indicates expression of whole proliferative populations. The bottom row indicates expression of each individual proliferative generation. The y-axis is normalized proliferation. FIG. 19A is a graph of CD3 proliferation for various MSC:PBMC ratios. FIG. 19B is a graph of CD3 generations for the various MSC:PBMC ratios. FIG. 19C is s a graph of CD4 proliferation. FIG. 19D is a graph of CD4 generations. FIG. 19E is a graph of CD8 proliferation. FIG. 19F is a graph of CD8 generations. In FIGS. 19A-19F, St=stim, Ct=control, A=1:10, B=1:20, C=1:100, D=1:200, E=1:1000, F=1:2000.

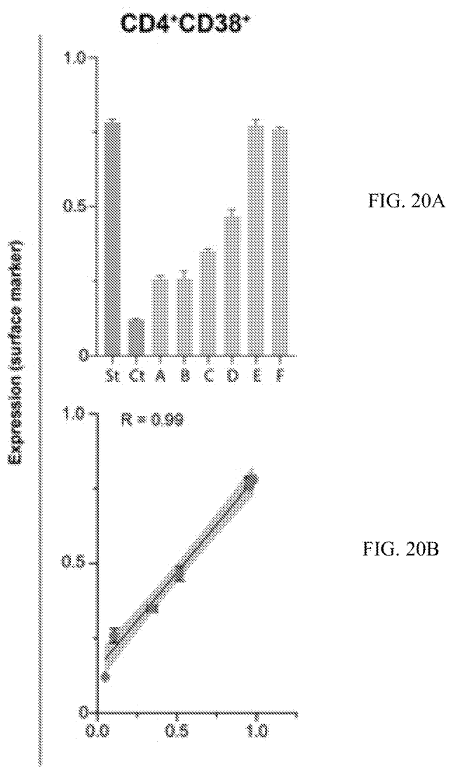

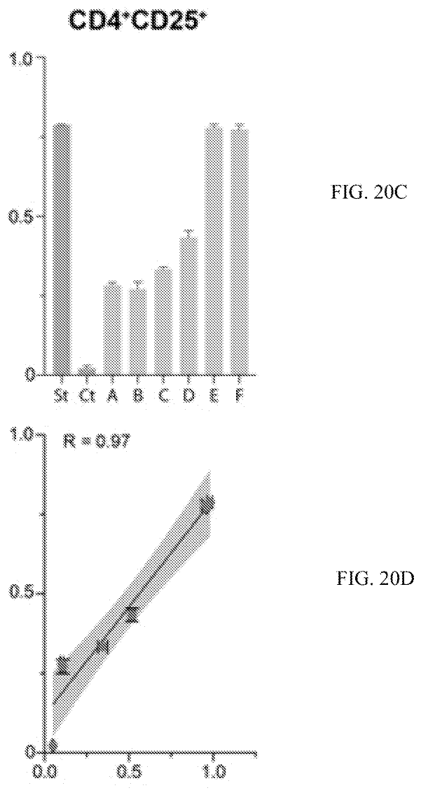

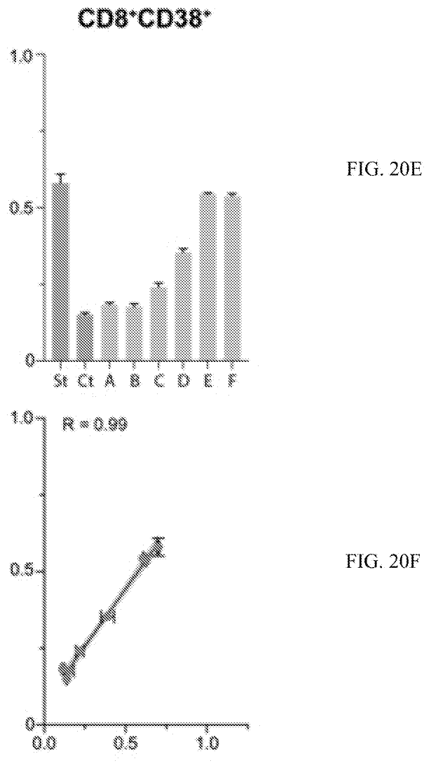

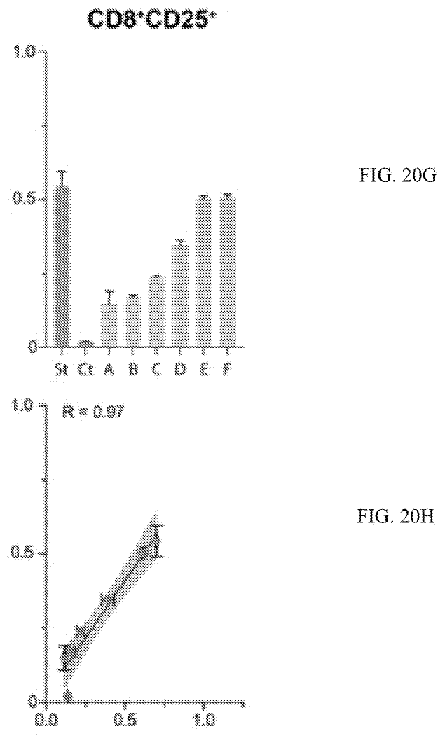

FIGS. 20A-20H illustrate flow cytometry data from an MSC:PBMC co-culture experiment. The top row (including FIGS. 20A, 20C, 20E, and 20G) indicates expression of whole proliferative populations. The bottom row (including FIGS. 20B, 20D, 20E, and 20F) indicates expression of each individual proliferative generation. The x-axis is normalized proliferation and the y-axis is surface marker expression level. FIG. 20A is a graph of CD4 proliferation and CD38 expression for various MSC:PBMC ratios. FIG. 20B is a graph of CD4 proliferation and CD38 expression showing high linearity/correlation. FIG. 20C is a graph of CD4 proliferation and CD25 expression for various MSC:PBMC ratios. FIG. 20D is a graph of CD4 proliferation and CD25 expression showing high linearity/correlation. FIG. 20E is a graph of CD8 proliferation and CD38 expression for various MSC:PBMC ratios. FIG. 20F is a graph of CD8 proliferation and CD38 expression showing high linearity/correlation. FIG. 20G is a graph of CD8 proliferation and CD25 expression for various MSC:PBMC ratios. FIG. 20H is a graph of CD8 proliferation and CD38 expression showing high linearity/correlation. In FIGS. 20A-20F20H, St=stim, Ct=control, A=1:10, B=1:20, C=1:100, D=1:200, E=1:1000, F=1:2000.

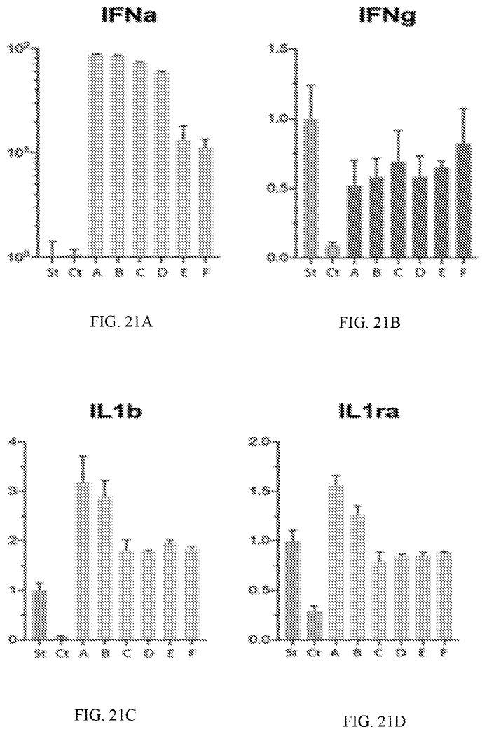

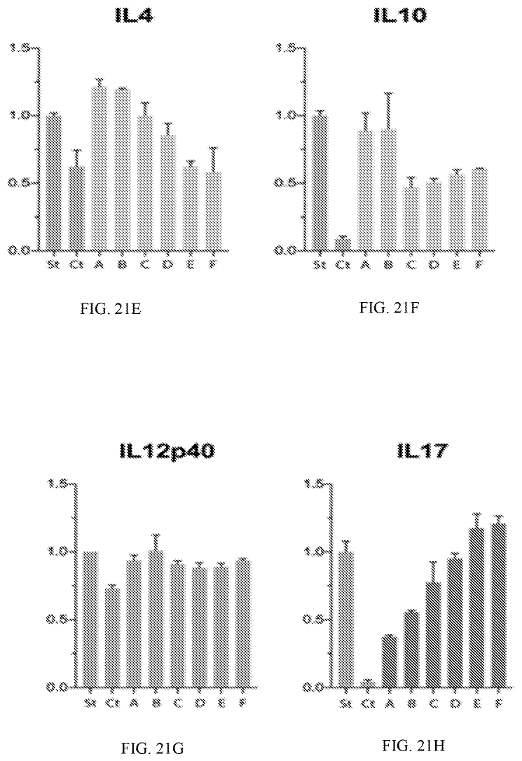

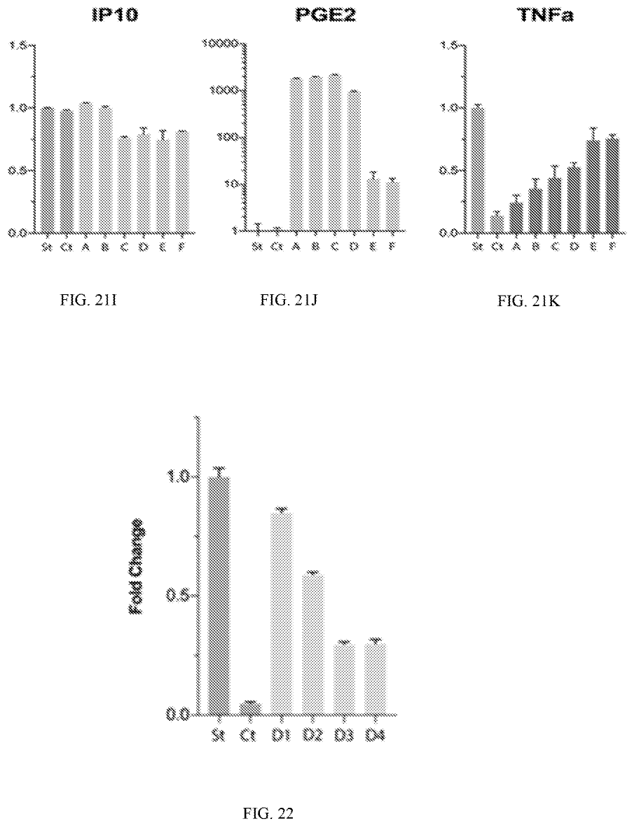

FIGS. 21A-21K illustrate multiplex dose response of secreted cytokines. FIG. 21A illustrates the response of IFNa for various MSC:PBMC ratios. FIG. 21B illustrates the response of INFg. FIG. 21C illustrates the response of IL1b. FIG. 21D illustrates the response of IL1ra. FIG. 21E illustrates the response of IL4. FIG. 21F illustrates the response of IL10. FIG. 21G illustrates the response of IL12p40. FIG. 21H illustrates the response of IL17. FIG. 21I illustrates the response of IP10. FIG. 21J illustrates the response of PGE2. FIG. 21K illustrates the response of TNFa. In FIGS. 21A-21K, St=stim, Ct=control, A=1:10, B=1:20, C=1:100, D=1:200, E=1:1000, F=1:2000.

In FIGS. 19A-21K, St=stim, Ct=control, A=1:10, B=1:20, C=1:100, D=1:200, E=1:1000, F=1:2000.

FIG. 22 is a graph of normalized proliferation of PBMC versus time of exposure to MSCs. PBMC proliferation was attained through stimulation with ConA and IL2 for a period of 4 days. MSC Transwell® inserts were removed after 1, 2, and 3 days co-culture initiation to time duration. Proliferation was measured through flow cytometry and CFSE staining. Significant duration time to therapeutic MSCs was required to elicit a full response. It was found that 3 out of 4 days was necessary at a potent MSC dose. There is noticeable immunosuppression at days 1 and 2, however at a significantly reduced level.

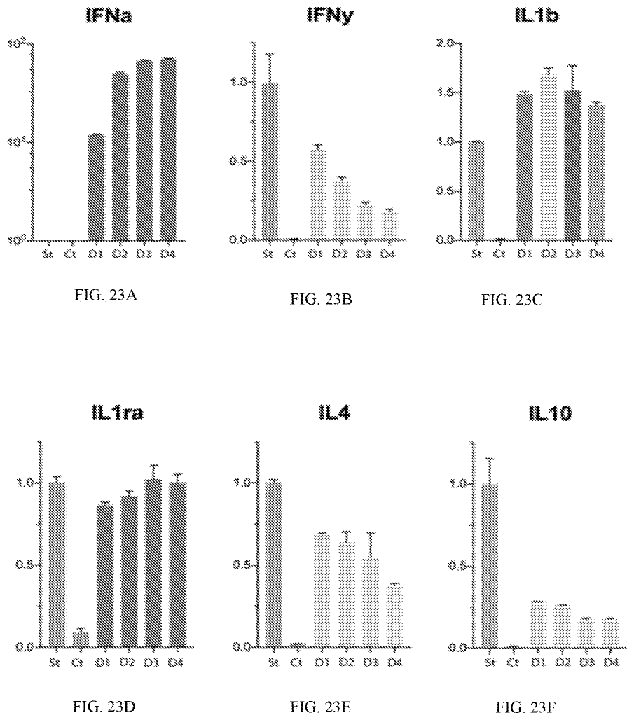

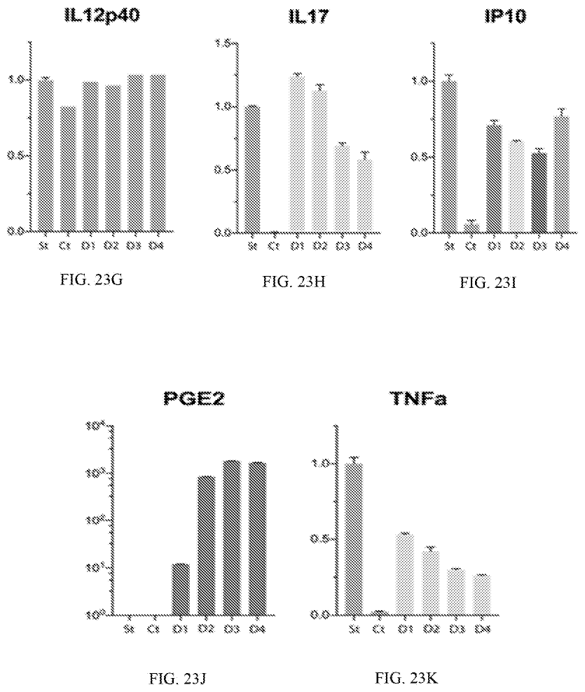

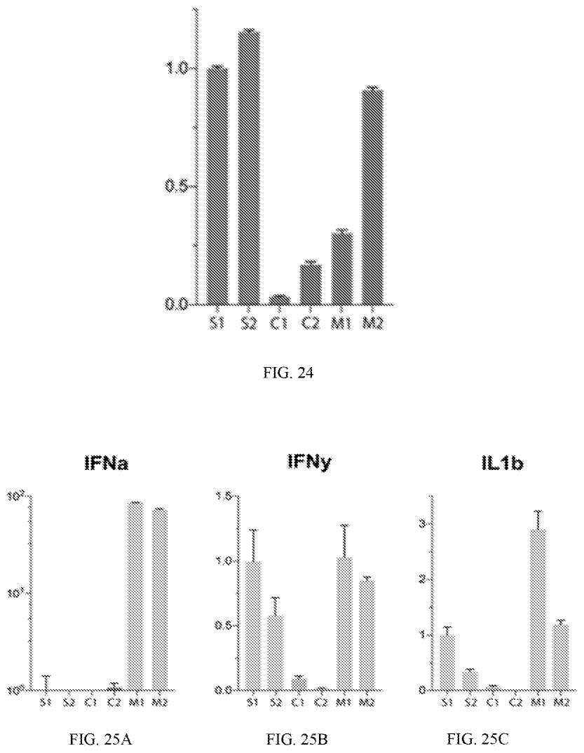

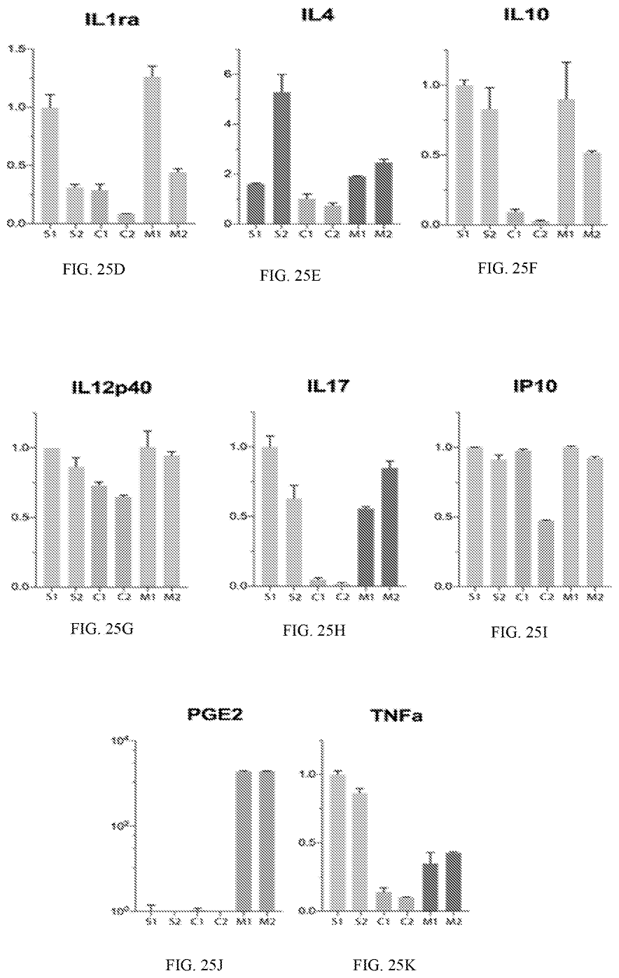

FIGS. 23A-23K are bar graphs of normalized cytokine secretion versus time of exposure to MSCs. PBMC proliferation was attained through stimulation with ConA and IL2 for a period of 4 days. MSC Transwell inserts were removed after 1, 2, and 3 days co-culture initiation to time duration. Proliferation was measured through flow cytometry and CFSE staining. Investigating cytokine profiling, concordant associations were seen, as in previous sections. Longer exposure to MSCs resulted in greater suppression of inflammatory cytokines an vice versa for short exposures. FIG. 23A illustrates an increase with prolonged MSC exposure for IFNa. FIG. 23B illustrates a decrease with prolonged MSC exposure for INFy. FIG. 23C illustrates no significant change with prolonged MSC exposure for IL1b. FIG. 23D illustrates a slight increase with prolonged MSC exposure for IL1ra. FIG. 23E illustrates a decrease with prolonged MSC exposure for IL4. FIG. 23F illustrates a decrease with prolonged MSC exposure for IL10. FIG. 23G illustrates no significant change with prolonged MSC exposure for IL12p40. FIG. 23H illustrates a decrease with prolonged MSC exposure for IL17. FIG. 23I illustrates no significant change with prolonged MSC exposure for IP10. FIG. 23J illustrates an increase with prolonged MSC exposure for PGE2. FIG. 23K illustrates a decrease with prolonged MSC exposure for TNFa.

FIG. 24 is a graph of normalized proliferation versus culture volume conditions. As shown in FIG. 24, volume is an implicit microenvironment factor that drives MSC potency. PBMC proliferation was attained through stimulation with ConA and IL2 for a period of 4 days. Proliferation was measured through flow cytometry and CFSE staining. It was found that doubling the volume of the co-culture greatly reduced the potency of MSCs.

FIGS. 25A-25K are bar graphs of normalized cytokine secretion versus culture volume conditions. As shown in FIGS. 25A-25, volume is an implicit microenvironment factor that drives MSC potency. PBMC proliferation was attained through stimulation with ConA and IL2 for a period of 4 days. Proliferation was measured through flow cytometry and CFSE staining. Clearly, this effect is driven by a pharmacologic effect through the dilution of intrinsic factors. Interestingly, no effect was seen on MSC predominant factors, indicating a lack of efficacy of these specific factors and a potential autocrine regulation due to similarly secreted levels at both volumes. Predominant PBMC factors were elevated at 2×, indicating a lack of MSC effect. This may also reflect a lack of sufficient initial cytokine levels to license MSCs.

In FIGS. 24-25K, S1=stim 1×vol, S2=stim 2×vol, C1=ctrl 1×vol, C2=ctrl 2×vol, M1=co-culture 1×vol, M2=co-culture 2×vol

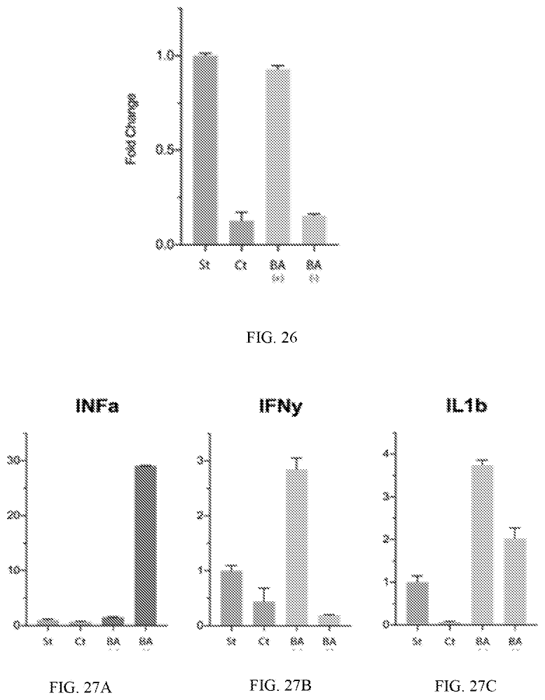

FIG. 26 shows critical MSC:PBMC cross communication, as shown by the use of a protein transport inhibitor. Either PBMCs or MSCs were treated for 24 hours with Brefeldin A prior to the start of co-culture. PBMC proliferation was attained through stimulation with ConA and IL2 for a period of 4 days. Proliferation was measured through flow cytometry and CFSE staining. As expected, it was found that treatment of MSCs with BA abolished therapeutic function.

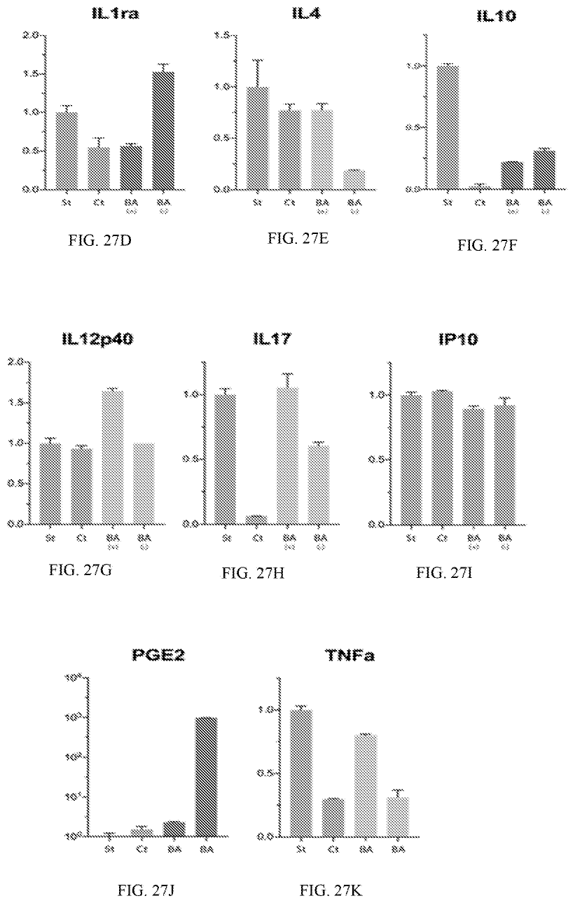

FIGS. 27A-K illustrate that MSC:PBMC cross communication is critical, as shown by the use of a protein transport inhibitor. Either PBMCs or MSCs were treated for 24 hours with Brefeldin A prior to the start of co-culture. PBMC proliferation was attained through stimulation with ConA and IL2 for a period of 4 days. Proliferation was measured through flow cytometry and CFSE staining. As expected, the results show greatly diminished MSC secreted factors. Significant increases in pro-inflammatory factors are also observed, which fall directly in line with MSC immunosuppressive function.

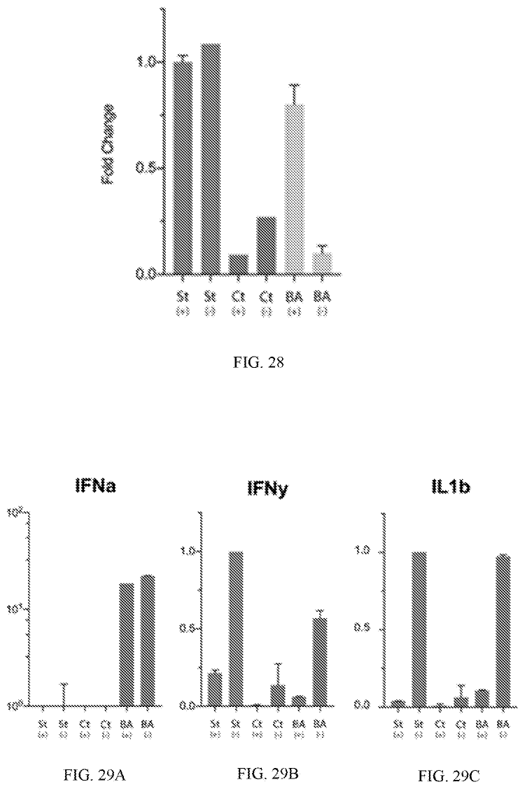

FIG. 28 shows the results of Brefeldin A treatment on PBMC:MSC co-culture. Either PBMCs or MSCs were treated for 24 hours with Brefeldin A prior to the start of co-culture. PBMC proliferation was attained through stimulation with ConA and IL2 for a period of 4 days. Proliferation was measured through flow cytometry and CFSE staining. It was further found that BrefA treatment of PBMCs results in greatly diminished MSC function, which supports the need for MSC licensing.

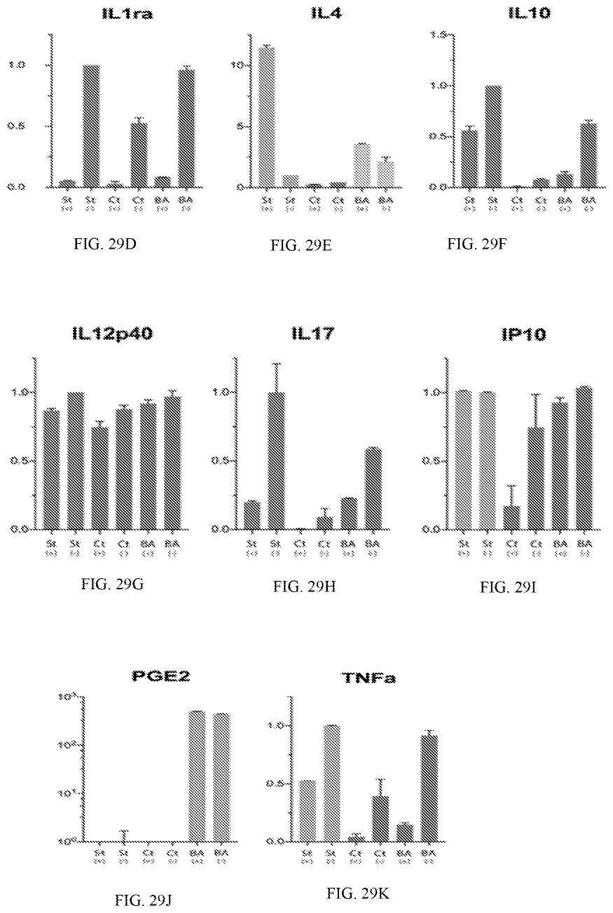

FIGS. 29A-K show the results of Brefeldin A treatment on PBMC:MSC co-culture. Either PBMCs or MSCs were treated for 24 hours with Brefeldin A prior to the start of co-culture. PBMC proliferation was attained through stimulation with ConA and IL2 for a period of 4 days. Proliferation was measured through flow cytometry and CFSE staining. Bar graphs of normalized cytokine secretion versus Brefeldin A conditions. Significant suppression of PBMC predominant factors was found, which likely results in insufficient MSC licensing leading to a lack of immunosuppression.

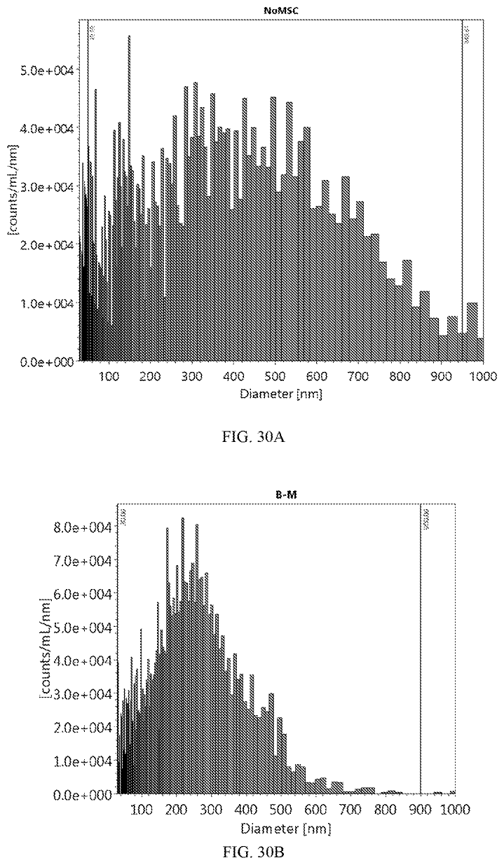

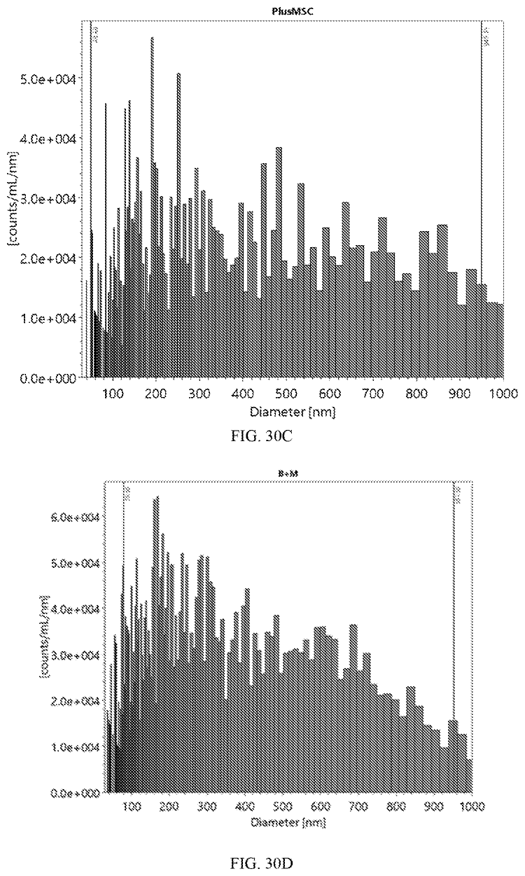

FIGS. 30A-30D show secreted particle size distribution from various bioreactor culture conditions. FIG. 30A illustrates a particle count of stimulated PBMC culture. FIG. 30B illustrates a particle count of stimulated PMBC culture. FIG. 30C illustrates a particle count from stimulated PBMC:MSC culture. FIG. 30D illustrates a particle count from stimulated PBMC:MSC culture. NoMSC=stimulated PBMCs; B-M=stimulated PBMCs; PlusMSC=stimulated PBMCs+MSCs; B+M=stimulated PBMCs+MSCs.

### Example 3. Stop-Flow Cultures

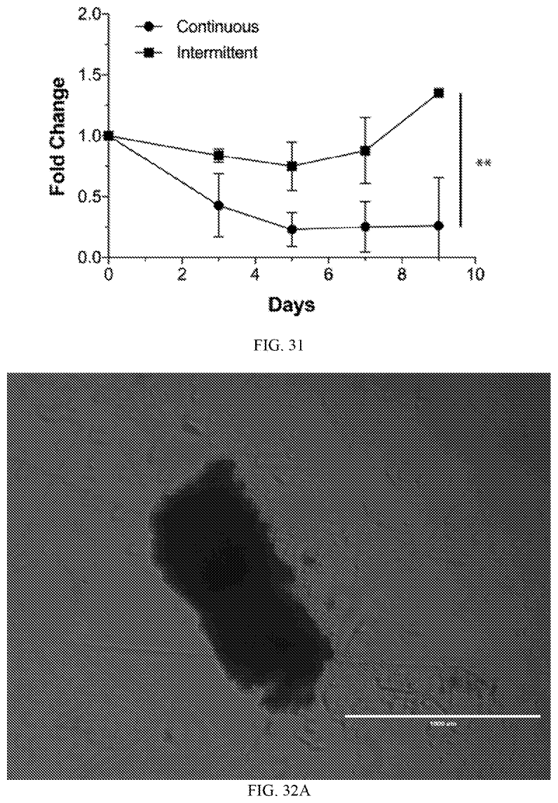

PBMCs (Stop & Continuous_3, FIG. 31) and purified T-Cells (Continuous_1 & Continuous 2, FIG. 31) were stimulated with 50 ng/mL CD3, 50 ng/mL CD28, and 50 ng/mL IL-2 in a gas permeable cell culture bag for up to 336 hours (14 days). Culture conditions were in a standard culture incubator at 37C and 5% CO2 using RPMI 1640 and 10% FBS. Recirculating continuous flow was held at 50-100 mLs/min for the entire duration of the continuous flow culture. Stop-flow cultures were held in static culture except for a duration of 5 minutes at 50-100 mls/min on the day of trypan blue exclusion cell counting (blue line/dots).

As shown in FIGS. 32A-B, static culture enabled aggregation whereas the addition of a brief pulse of flow caused a shear-induced dispersion of cells. T his was found to be beneficial because it 1) attenuated large aggregates, which may become nutrient diffusion limited, thereby resulting in non-ideal cell culture conditions; and 2) allowed for cell enumeration, which is not possible with large, intact cell aggregates.

### Example 4. Magnetic Pumps for Stop-Flow Cultures

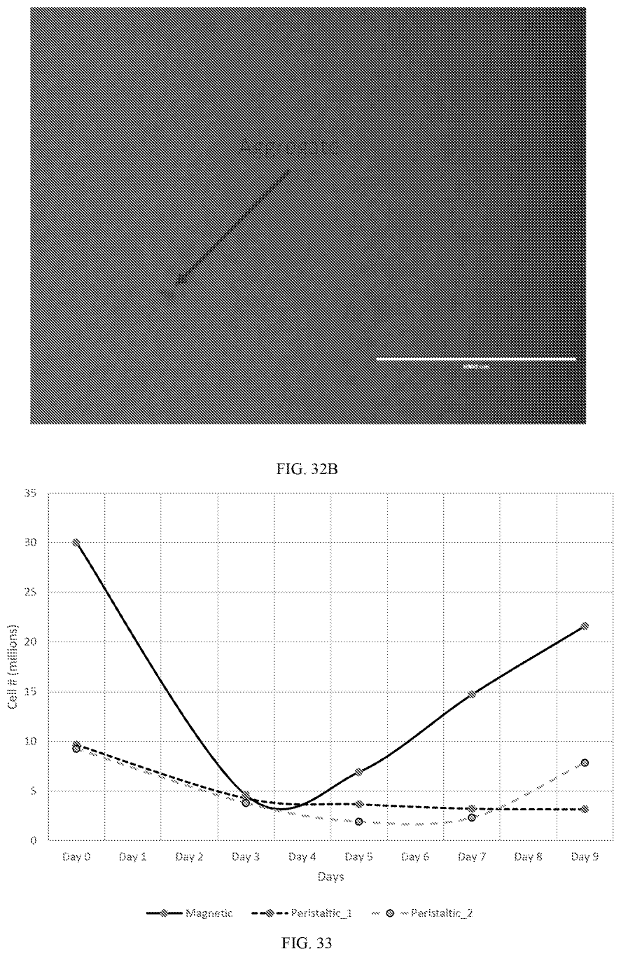

PBMCs (Magnetic, FIG. 33) were stimulated with 50 ng/mL CD3, 50 ng/mL CD28, and 50 ng/mL IL-2 and PBMCs (Peristaltic, FIG. 33) were stimulated with 5 μg/mL PHA-L and 100 ng/mL IL-2 in a gas permeable cell culture bag for up to 240 hours (10 days). Magnetic Flow was introduced and compared to peristaltic pump flow. Cell counts were taken from the cell culture bag at various time points. Yields were found to be greater after the 10 day period using continuous magnetic flow. Significant debris was observed in peristaltic flow which is indicative of cell death due to mechanical disruption associated with the peristaltic mechanism.

### Example 5. miRNA Expressed in PBMCs Exposed to MSC Exosomes and Pure MSC Exosomes

PBMCs were exposed to MSC exosomes over a 4-day culture period in a hollow-fiber bioreactor. MSCs were seeded on the extraluminal surface while PBMCs were allowed to flow within the intraluminal space at a flow rate of 50-100 mLs/min. Cultures were stimulated to induce an inflammatory environment.

The results of miRNA expressed in MSC exosomes and in PBMCs that were exposed to the MSC exosomes over the culture period are shown in Table 3. The numbers shown in both columns represent MFIs (mean fluorescent intensities) from the assay and are indicative of the amount of miRNA present in the sample. Supernatant: concentrated sample of bioreactor experiment with only MSCs in the system. Pellet: PBMC pellet sample of bioreactor experiment with both MSCs and PBMCs in the system.

TABLE 3
miRNA Expressed in reprogrammed PBMCs

| miRNA | Pellet | Supernatant | miRNA | Pellet | Supernatant |
|---|---|---|---|---|---|
| hsa-mir-199a-5p | 0 | 20.32 | hsa-mir-28-5p | 37.64 | 1.35 |
| hsa-mir-381-3p | 0 | 47.98 | hsa-mir-98-5p | 41.22 | 1.5 |
| hsa-mir-494-3p | 0 | 149.22 | hsa-mir-378c | 42.58 | 4.29 |
| hsa-mir-382-5p | 0.02 | 57.24 | hsa-mir-422a | 44.42 | 3.38 |
| hsa-mir-495-3p | 0.2 | 60.03 | hsa-mir-342-5p | 46.96 | 0.23 |
| hsa-mir-485-3p | 0.22 | 11.03 | hsa-mir-484 | 51.14 | 84.97 |
| hsa-mir-299-3p | 0.28 | 10.22 | hsa-mir-181c-5p | 51.71 | 0.78 |
| hsa-mir-224-5p | 0.28 | 22.5 | hsa-mir-34a-5p | 53.3 | 414.81 |
| hsa-mir-409-3p | 0.31 | 26.83 | hsa-mir-151a-5p | 55.3 | 38.18 |
| hsa-mir-376a-3p | 0.42 | 22.57 | hsa-mir-425-5p | 60.29 | 11.35 |
| hsa-mir-376c-3p | 0.44 | 92.03 | hsa-mir-26b-5p | 60.51 | 0.57 |
| hsa-mir-4516 | 0.45 | 13.75 | hsa-let-7d-3p | 61.74 | 46.67 |
| hsa-mir-337-5p | 0.52 | 77.94 | hsa-mir-194-5p | 62.37 | 1.15 |
| hsa-mir-543 | 0.56 | 23.56 | hsa-mir-223-3p | 62.81 | 0.78 |
| hsa-mir-10b-5p | 0.69 | 22.27 | hsa-mir-181d-5p | 70.76 | 3.01 |
| hsa-mir-323a-3p | 0.71 | 54.09 | hsa-mir-186-5p | 77.86 | 30.82 |
| hsa-mir-122-5p | 0.78 | 251.88 | hsa-mir-197-3p | 84.52 | 62.78 |
| hsa-mir-323b-3p | 0.8 | 40.52 | hsa-mir-363-3p | 91.08 | 1.48 |
| hsa-mir-329-3p | 0.86 | 32.29 | hsa-mir-130a-3p | 91.57 | 954.73 |
| hsa-mir-127-3p | 0.91 | 27.97 | hsa-mir-18b-5p | 102.19 | 6.63 |
| hsa-mir-214-3p | 0.94 | 359.08 | hsa-mir-378a-3p | 104.44 | 9.26 |
| hsa-mir-143-3p | 1.08 | 470.49 | hsa-mir-132-3p | 105.74 | 324.76 |
| hsa-mir-145-5p | 1.12 | 110.54 | hsa-mir-18a-5p | 112.5 | 4.34 |
| hsa-mir-154-5p | 1.38 | 78.33 | hsa-mir-185-5p | 114.64 | 82.22 |
| hsa-mir-708-5p | 1.4 | 17.82 | hsa-mir-130b-3p | 127.86 | 275.28 |
| hsa-mir-10a-5p | 1.46 | 28.54 | hsa-mir-181b-5p | 134.34 | 11.93 |
| hsa-let-7b-3p | 1.62 | 33.02 | hsa-mir-15b-5p | 135.08 | 28.03 |
| hsa-mir-345-5p | 2.25 | 22.3 | hsa-mir-181a-5p | 149.25 | 4.56 |
| hsa-mir-193a-3p | 2.66 | 28.16 | hsa-mir-106b-5p | 162.89 | 13.47 |
| hsa-mir-199a-3p | 2.67 | 303.43 | hsa-mir-423-5p | 201.99 | 444.65 |
| hsa-mir-138-5p | 3.37 | 152.26 | hsa-let-7e-5p | 222.1 | 2.4 |
| hsa-mir-30a-5p | 3.49 | 22.81 | hsa-mir-222-3p | 222.58 | 1664.5 |
| hsa-mir-410-3p | 4.29 | 12.51 | hsa-mir-27b-3p | 259.74 | 534.35 |
| hsa-mir-193b-5p | 5.33 | 62.35 | hsa-mir-1260b | 269.4 | 14.27 |
| hsa-mir-486-5p | 5.66 | 12.88 | hsa-mir-320d | 291.41 | 938.09 |
| hsa-mir-497-5p | 5.82 | 30.2 | hsa-mir-221-3p | 301.44 | 3053 |
| hsa-mir-22-5p | 6.3 | 26.06 | hsa-mir-23b-3p | 315.34 | 1117.18 |
| hsa-mir-30d-5p | 8.45 | 23.9 | hsa-let-7f-5p | 339.87 | 1.77 |
| hsa-mir-328-3p | 10.74 | 23.94 | hsa-mir-15a-5p | 348.38 | 55.82 |
| hsa-mir-141-3p | 10.86 | 0.43 | hsa-mir-30b-5p | 353.47 | 7 |
| hsa-mir-106b-3p | 11.06 | 0.91 | hsa-mir-195-5p | 354.57 | 91.86 |
| hsa-mir-100-5p | 11.55 | 1216.13 | hsa-mir-1260a | 371.81 | 5.13 |
| hsa-mir-361-3p | 11.95 | 0.17 | hsa-mir-128-3p | 397.43 | 351.92 |
| hsa-mir-18a-3p | 12.41 | 0.84 | hsa-mir-26a-5p | 401.17 | 2.13 |
| hsa-mir-103a-2-5p | 12.8 | 5.81 | hsa-mir-30c-5p | 410.98 | 8.21 |
| hsa-mir-140-5p | 13.27 | 2.21 | hsa-mir-29c-3p | 455.28 | 41.39 |
| hsa-mir-301b-3p | 13.37 | 4.18 | hsa-mir-146b-5p | 499.3 | 168.87 |
| hsa-mir-21-3p | 13.91 | 26.61 | hsa-mir-342-3p | 502.32 | 49.63 |
| hsa-mir-33a-5p | 14.92 | 0.75 | hsa-mir-320c | 505.02 | 1976.02 |
| hsa-mir-574-3p | 15.12 | 228.9 | hsa-mir-25-3p | 513.97 | 228.78 |
| hsa-mir-532-3p | 15.46 | 6.57 | hsa-mir-146a-5p | 580.09 | 609.01 |
| hsa-mir-320e | 15.7 | 174.88 | hsa-mir-27a-3p | 621.08 | 1061.17 |
| hsa-mir-296-5p | 15.93 | 5.64 | hsa-let-7i-5p | 632.61 | 100.52 |
| hsa-mir-454-3p | 16.35 | 0.57 | hsa-let-7c-5p | 696.18 | 46.44 |
| hsa-mir-744-5p | 16.93 | 0.19 | hsa-mir-191-5p | 699.32 | 19.43 |
| hsa-mir-210-3p | 17.64 | 48.78 | hsa-mir-107 | 738.24 | 31.39 |
| hsa-mir-7-l-3p | 18.39 | 2.86 | hsa-mir-23a-3p | 779.07 | 2542.54 |
| hsa-mir-301a-3p | 18.82 | 1.54 | hsa-mir-19a-3p | 788.1 | 121.52 |
| hsa-mir-28-3p | 18.92 | 5.24 | hsa-let-7b-5p | 831.9 | 213.03 |
| hsa-mir-99a-5p | 18.99 | 143.13 | hsa-mir-24-3p | 903.35 | 1009.97 |
| hsa-mir-34c-5p | 20.01 | 4.01 | hsa-mir-93-5p | 956.77 | 94.28 |
| hsa-mir-124-3p | 20.21 | 0.37 | hsa-mir-103a-3p | 1089.79 | 26.83 |
| hsa-mir-652-3p | 20.32 | 6.3 | hsa-mir-320b | 1103.04 | 2839.16 |
| hsa-mir-660-5p | 21.62 | 21.2 | hsa-mir-22-3p | 1118.93 | 4166.45 |
| hsa-mir-532-5p | 22.04 | 17.2 | hsa-mir-29a-3p | 1135.66 | 1121.91 |
| hsa-mir-31-5p | 22.05 | 13.88 | hsa-mir-92b-3p | 1196.6 | 875.88 |
| hsa-mir-142-3p | 24.59 | 0.24 | hsa-let-7a-5p | 1238.78 | 7.3 |
| hsa-mir-193a-5p | 25.12 | 604.82 | hsa-mir-320a | 1253.01 | 3123.07 |
| hsa-mir-324-5p | 28.3 | 0.7 | hsa-mir-19b-3p | 1560.19 | 233.05 |
| hsa-mir-148b-3p | 28.32 | 29.8 | hsa-mir-29b-3p | 1570.2 | 40.63 |
| hsa-mir-192-5p | 28.65 | 2.98 | hsa-mir-106a-5p | 1719.7 | 119.94 |
| hsa-mir-200c-3p | 28.85 | 0.5 | hsa-mir-17-5p | 1887.5 | 181.65 |
| hsa-mir-423-3p | 28.86 | 7.87 | hsa-let-7d-5p | 2026.76 | 4.67 |
| hsa-mir-17-3p | 29.55 | 0.26 | hsa-mir-20b-5p | 2406.61 | 162.75 |
| hsa-mir-148a-3p | 31.18 | 42.42 | hsa-let-7g-5p | 2429.38 | 19.62 |
| hsa-mir-99b-5p | 31.18 | 66.67 | hsa-mir-155-5p | 2780.01 | 3.15 |
| hsa-mir-4500 | 32.1 | 1.88 | hsa-mir-21-5p | 2811.54 | 3033.49 |
| hsa-mir-152-3p | 32.24 | 75.37 | hsa-mir-92a-3p | 3557.75 | 2314.97 |
| hsa-mir-193b-3p | 32.45 | 157.8 | hsa-mir-20a-5p | 4129.65 | 743.45 |
| hsa-mir-151b | 36.1 | 36.17 | hsa-mir-150-5p | 5380.83 | 0.89 |
| hsa-mir-361-5p | 36.91 | 10.63 | hsa-mir-16-5p | 7068.43 | 3797.99 |

### Example 6. Transfer of Lentiviral Particles by Producer Cells in a Transwell Engineering System

Lentiviruses are derived from human immunodeficiency virus (HIV) which allows them to be effective delivery systems. However, prior to using them to deliver genetic materials to cells of interest, the normal course of production of these viruses involves a lengthy collection and quantification process. The ability for producer HEK293T cells to simultaneously produce lentiviral particles and transduce (infect) target cells in a transwell system, which negates the need for a separate viral collection and quantification process, was demonstrated. For each experiment, variations in HEK293T and target cell type densities as well as transwell insert porosities were assessed to identify key relationships between particle production rate and infection kinetics for adherent and suspension cell types. The experiments successfully showed the ability to engineer human PBMCs under the control of this system in under six days with an RFP construct. These studies suggest the capability to combine and more closely automate the transfection/transduction process in order to facilitate well-timed and cost-effective transduction of target human cell types. Such studies can provide for transition into improved manufacturing systems for viral production and subsequent cell therapy engineering.

The field of genetic engineering and therapy has utilized three primary viral vector systems (Adeno-associated viruses (AAV), y-retroviruses, and lentiviruses) with increasing success in a number of disorders ranging from X-Linked Severe Combined Immunodeficiency (SCID-X1), Hemophilia B, and hematological malignancies using Chimeric Antigen Receptor (CAR)-T cells (Rogers et al., 2015; Hacein-Bey-Abina et al., 2014; Porter et al., 2011). Lentiviral vectors in particular have also been used elsewhere in treatment of rare diseases, including primary immunodeficiencies and neurodegenerative storage diseases (Mukherjee et al., 2013; Aiuti et al., 2013; Cartier et al., 2009; Biffi et al., 2013). Lentiviruses have been used and optimized over the past several decades and can be a preferred viral vector system due to their ability to transduce both dividing and nondividing cells, their safer integration profile and their ability to be produced at high vector titer (Merten et al., 2016). Lentiviral vectors attribute their powerful infectivity to being based off of the HIV-1 backbone, as well as through pseudotyping with the VSV-G envelope protein which allows for improved tropism and transfer of genetic material through direct contact between viral vector and target cell surface for the majority of mammalian cell types (Durand et al., 2011; Farley et al., 2007).

Normal methods of lentiviral vector particle production at a small scale involve three or four plasmid addition to a 2D culture system of HEK293T cells at >90% confluency, followed by a collection process of cell culture supernatant that is further purified, quantified and stored at −80° C. (Ausubel et al., 2012). As shown in FIG. 36, the process from the initial seeding of the HEK293T cells until the time at which they can be used for subsequent transduction of a target cell type, takes on the order of 7-10 days depending on preferred quantification method used (Ausubel et al., 2012; Geraerts et al., 2006). As more clinical trials using lentiviral vectors receive clinical approval, transition to routine, large-scale manufacturing methods for cell therapies that limit the need for lengthy production times, minimization of reagent waste and extensive hands-on manipulation are desirable (Merten et al., 2016; Geraerts et al., 2006; Sheu et al., 2015; Gandara et al., 2018; Merten et al., 2010). With the current system used, viral particles are not at their peak titer during transfection because the processes of transfection and transduction are treated as two distinct events, meaning virus is not immediately used to deliver genes of interest. In contrast, a single, low manipulation system that allowed for production of particles and immediate transduction of a target cell can minimize the need for a separate viral collection and processing step. The current approach to production of lentivirus and gene delivery to cells calls for optimization to maximize titer and quantity of virus produced while maintaining end product (target cell) sterility during the manufacturing process. The importance of this system is to limit the possibility of contamination while allowing for a more streamline transition to a large-scale, closed system cell engineering manufacturing platform.

This study showed the potential of a one-step lentiviral particle production and target cell transduction system through incorporation of a transwell based set up. Manipulation of a transwell based system can provide further insight into target cell therapy product parameters, where control of particle output and subsequent multiplicity of infection (MOI) can be determined through variation of system parameters such as insert porosity and cell density. The data collected herein suggest that an optimal range for particle output and subsequent infection can be determined for both adherent and suspension cell types in a transwell based system to match demands of a cell therapy product. Furthermore, combination of the two key processes of transfection and transduction can address many of the current issues associated with timelier and cost-effective cell manufacturing methods using lentiviral vectors (Milone et al., 2018).

Lentiviral Particle Secreting HEK293T Cells can Infect Target Cells in Transwell System

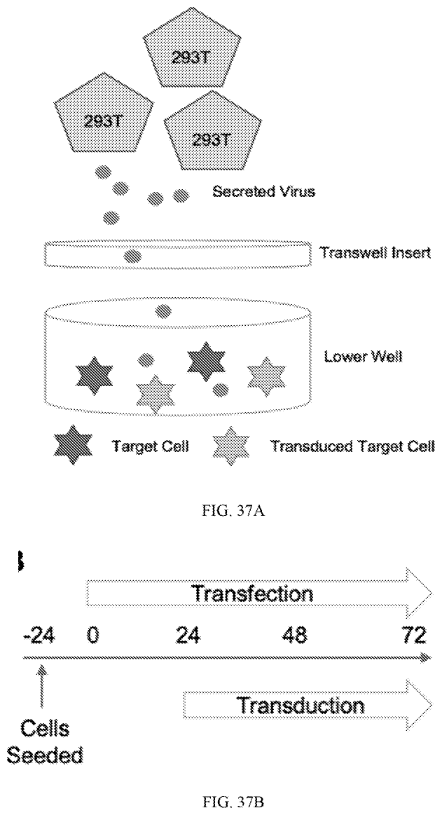

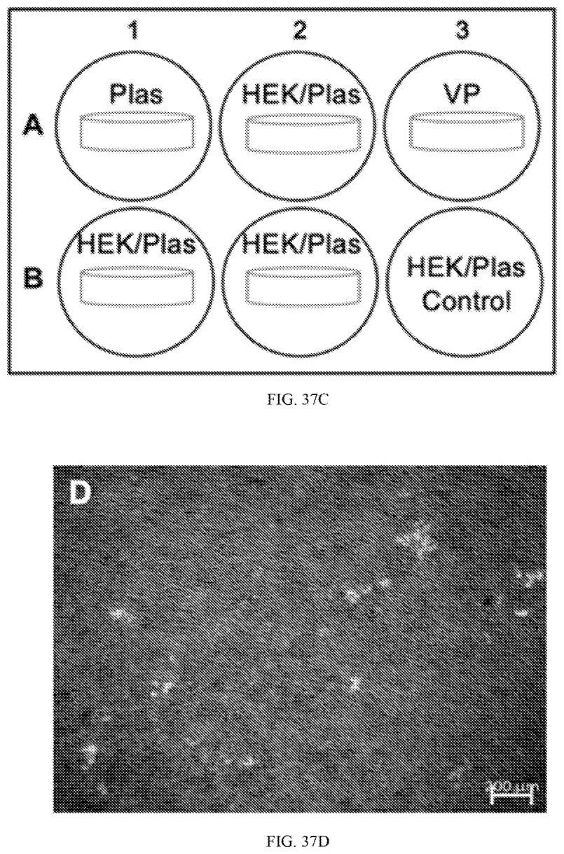

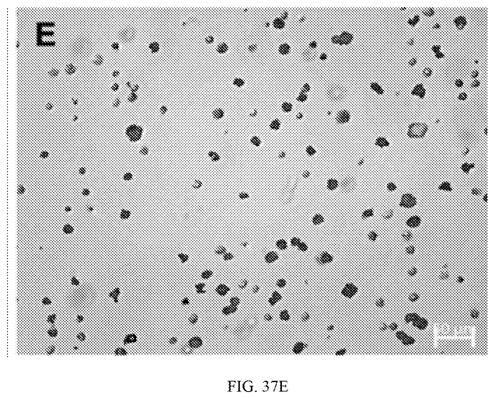

HEK293T cells, al cell line for production of lentiviral particles, were seeded at 45% confluency in 0.4 μm inserts 24 hours prior to addition of lentiviral particle packaging plasmids in order to reach an optimal density of 90% at start of transfection. Initial experiments used adherent pancreatic cancer cells (patient 1319) and suspension Jurkat T cells to assess the feasibility of a transwell based system. Target cells were seeded 24 hours prior to plasmid addition at 1.8×105 cells/mL in a 6-well in order to reach a desired transduction confluency of 25-30% at time when HEK293T cells begin to produce lentiviral particles (between day 24 and 48 as shown in FIGS. 37A-E). After plasmid addition on day 0, plates were returned to incubator at 37° C. and 5% CO2. 24 hours later, media in transwell inserts was changed to collection media (DMEM/F12 only) following standard transfection protocol at which point HEK293T cells began producing progeny lentiviral particles (24-72 hours in FIGS. 37A-E). At 48 hours, 8 ug/mL polybrene transduction reagent was added to all plates, which were shaken at 600 rpm for 90 minutes at 25° C. to mimic the standard spinoculation protocol for suspension Jurkat T cells prior to being moved to an incubator for 8-12 hours. Afterwards, the media was changed to complete growth media for the target cell type in each well and allowed to grow for an additional 48 hours. Transduction in each well was then assessed using a ZEISS microscope for GFP expression shown below in FIGS. 27C-D corresponding to 1319 and Jurkat T cells respectively.

Varying Density of HEK293T and Target Jurkat T Cells

Once confirmation of transduction of target cells in a transwell was made, variations in seeding density for target cells and HEK293T cells was assessed. HEK293T cells were seeded a day prior to plasmid addition to reach target confluencies of 90%, 75%, 60%, 45% and 30% on the day of transfection. Jurkat T cells were seeded to reach an optimal density of 30% at the start of transduction in each well of the 6-well. Flow Cytometry (FACS) Analysis was used to determine the effect on HEK293T cell density in each insert on the transduction efficiency of target Jurkat T cells.

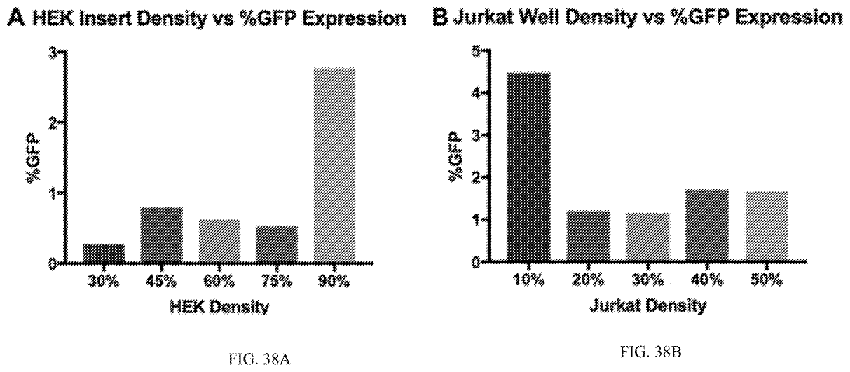

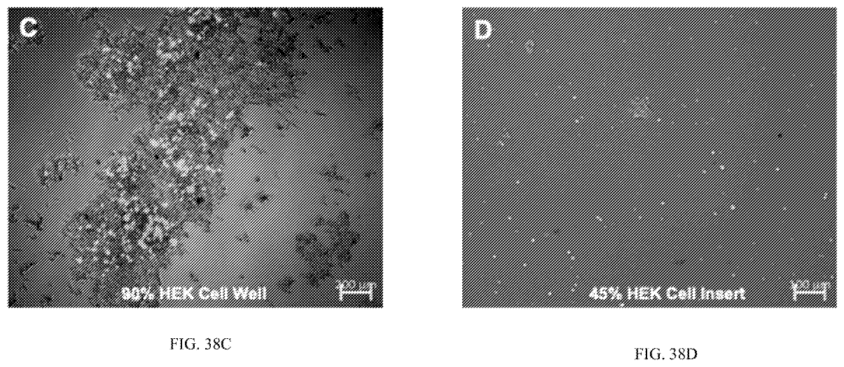

Jurkat T cell density in wells was also varied from 10%, 20%, 30%, 40% and 50% to assess effect on transduction efficiency, while HEK293T cells was maintained in the insert at the optimal 90% confluency. FIG. 38A compares the variation in HEK293T cell density and FIG. 38B compares the Jurkat T cell density variations.

HEK293T cells seeded at 90% confluency in the inserts at time of transfection showed the highest level of particle production and therefore subsequent transduction of target Jurkat T cells in the bottom well. Although we saw the most GFP positive cells, there were additionally HEK cells in the bottom well of the transwell, which is an undesired side effect, as shown in FIG. 38C. Therefore, for downstream applications a lower density, such as 45% HEK293T cells in the insert may serve more appropriate such as in FIG. 38D. Jurkat T cells initially seeded at 10% confluency prior to transduction showed the most GFP positive cells.

Variation in Transwell Insert Pore Size

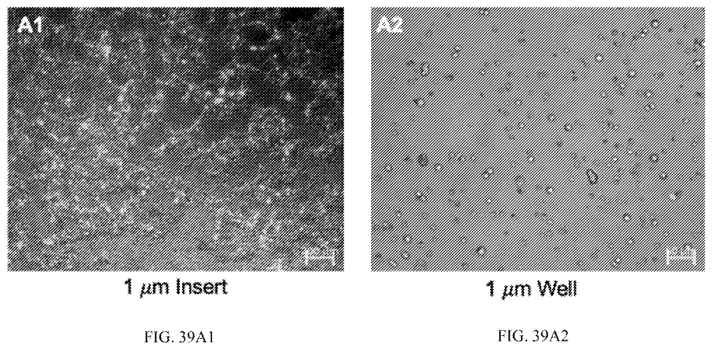

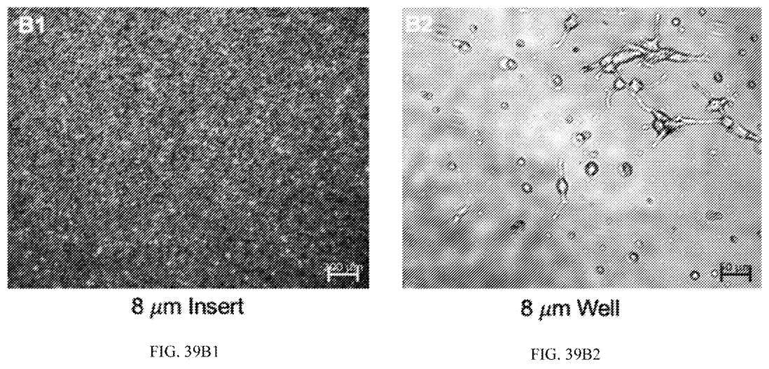

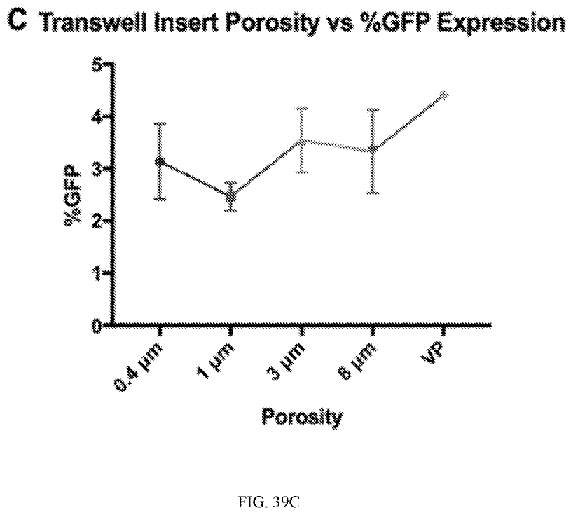

Another variable that was chosen to be manipulated was transwell insert pore size. Inserts of various pore sizes were used to assess the impact on transduction efficiency in a transwell system using HEK293T cells and Jurkat T cells at ideal transduction confluencies. Inserts were chosen that varied in porosity from 0.4, 1, 3 and 8 Flow cytometry analysis was used to determine transduction efficiency for each group. Results are shown in FIGS. 39A1-C.

For smaller pore sizes, such as 0.4 and 1 μm, it is likely that the viral particles are clogging the pores as they are simultaneously being produced which limits their ability to permeate and transduce target cells in the well. For the 8 μm, although there was a higher transduction efficiency seen, the larger pore size allowed for permeation of HEK293T cells through the membrane, which is not ideal.

Transduction of Human PBMCs

In order to assess the effectiveness of the transwell system to transduce a target human cell type, human peripheral blood mononuclear cells (PBMCs) from donor PM were stained with CellTrace™ CFSE Cell Proliferation stain prior to being seeded as described in the materials and methods section. Cells were stimulated 1 day prior to the start of transfection and 3 days prior to the start of transduction using PHA and IL-2. Flow cytometry was used to determine transduction efficiency as well a proliferation potential of PMBCs using the transwell based system. An RFP construct was used in order to distinguish transduction from proliferation flourescence.

Discussion

In this work, we investigated whether a transwell-based cell engineering system could allow HEK293T cells to simultaneously produce lentiviral particles and transduce target adherent 1319 pancreatic cancer cells and suspension Jurkat T cells and PBMCs in a one-system manner with minimal manipulation and reagant use.

Many of the current purification protocols involve the use of ultracentrifugation to concentrate the virus, but there is scale limitations with these methods, it is time consuming and oftentimes impurities are concentrated with the virus that can later cause an immunogenic reaction or interfere with transduction. A series of microfiltration steps are often employed, based on a series of decreasing pore size filters to minimize the decay of the particles associated with filtration. Tangential flow filtration (TFF) offers an alternative and can be applied to successfully concentrate and partially purify lentiviruses which is more scalable and efficient than previously discussed methods. In this report, several of these strategies are addressed in a one-approach system. To completely remove the effects associated with particle titer loss and damage due to a number of processing conditions currently implemented, HEK293T cells were used to continuously produce high titer lentiviral particles that are immediately in contact with target cells. Through use of a porous transwell membrane, the degree at which particles permeate through was able to be controlled and which porosities are responsible for allowing top HEK293T cells to also permeate through, which is undesirable and can be fixed, was identified.

Currently, viral collection for further downstream processing can be done at two distinct time points after plasmids are added to the HEK293T cells, and this occurs at 48 and 72 hours post transfection. During the collection process, many of the HEK293T cells are disturbed and leave the plate with the supernatant, also because they are easily removed once FBS is removed from the media and switched to collection (DMEM/F12 only media) and, therefore, the second round of collection can involve less than an ideal number of HEK293T cells that are producing viral particles. It can be recommended that, after 3 days, no further collections should be carried out because the supernatant can contain low volumes of poor quality viral particles. With this study, a method for continuous production of high quality lentivirus that can immediately come into contact with target cells for transduction was established.

In this study, successful transduction of human Jurkat T cells in the transwell based system was shown. Additionally, PBMCs were used to assess the ability for the transwell-based system to engineer target human cells that could be used for a downstream clinical setting.

Materials and Methods

Vector Production

Generation of lentiviral vectors was accomplished using Human 293T kidney fibroblast cell line (HEK293T) expressing a mutant version of the SV40 large T antigen (ATCC: CRL-3216) seeded at various densities within the cell inserts and wells depending on each experiment. The lentivirus transfer vector pLVEC-EFNB1-Fc-IRB was a kind gift from Rick Cohen containing a GFP or RFP reporter gene. Packaging plasmid psPAX2 and VSV-G expressing envelope plasmid pMD2.G were used for transfection in the presence of DMEM/F12 (Gibco)+10% FBS (Gibco)+1% Penicillin/Streptomycin (Gibco). Transductions took place in the presence of 8 ug/mL polybrene (Millipore Sigma) in corresponding media (detailed below) at 37° C. for specified lengths of time.

Cell Culture

HEK293T and 1319 cells were grown in DMEM/F12 (Gibco) supplemented with 10% FBS (Gibco) and 1% Pen/Strep (Gibco). Jurkat cells and PBMCs were cultured in RPMI-1640 media (Gibco) with 10% FBS (Gibco) and 1% Pen/Strep (Gibco). PBMCs were stained using CFSE Stain for proliferation and stimulated using PHA and IL-2.

PBMC Staining

PBMCs were stained using CellTrace™ CFSE for proliferation by combining 1 uL CFSE total for every 2 mL total of cells at a density between 2-4 million cells per mL. Cells were centrifuged down in a 15 mL conical tube at 1700 rpm for 5 minutes a resuspended in 5 mL of PBS. 3 uL CFSE was added and mixed and the tube was covered in foil and incubated at room temperature for 5 minutes. 2 mL of media was added on top of cell suspension in tube to reconstitute and tube was spun down again for 5 minutes. Cells were rinsed briefly with 5 mL of PBS once after removing supernatant from cell pellet, and cells were spun down a third time for 5 minutes. Replaced supernatant with media, took a cell count and plated the cells in a 6-well at 3.3×10\hat{ }6 cells in 3 mL.

PBMC Stimulation

PBMCs were stimulated to proliferate using phytohemagglutinin (PHA) and IL-2. Briefly, 3 uL PHA (1000 ng/mL) and 6 uL IL-2 (100 ng/mL) was added to each well and mixed gently on the same day HEK293T cells were seeded in each insert.

Transwell System

Transwell inserts were acquired from Grenier Bio-One and ranged in size from 8, 3, 1, and 0.4 μm pore size. Membranes were made from polyethylene terephthalate (PET) and were either translucent or transparent depending on size. The culture surface area for inserts was on average 456.05 mm².

Flow Cytometry

Lentiviral transduction of target cells was analyzed by GFP expression using a BDFacsCanto II (BD Biosciences) and Flow Jo software. 10,000 events per sample were collected for each experiment. Non-viable cells were excluded from analysis.

Fluorescent Microscopy

A ZEISS microscope was used to qualitatively analyze samples for expression of the green fluorescent protein (GFP) or red fluorescent protein (RFP) marker.

### Example 7. PBMC Proliferation

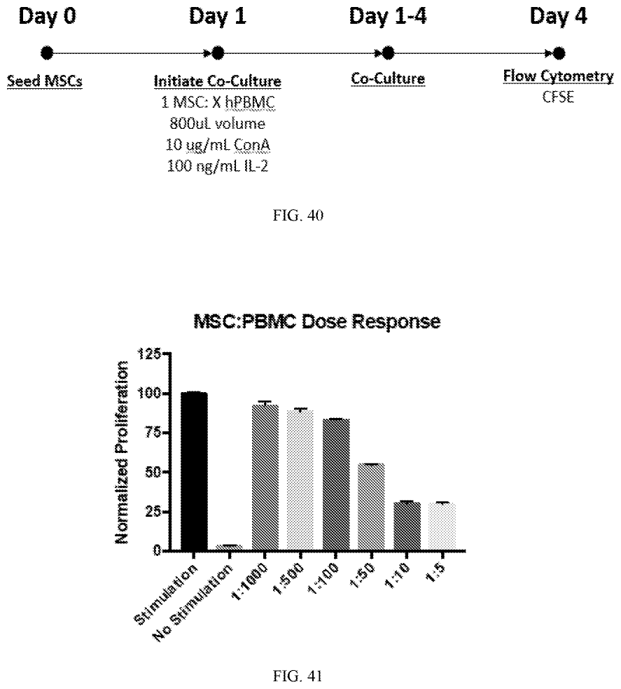

PBMC proliferation was attained through stimulation with ConA and IL2 for a period of 4 days. A schematic illustrating the study timeline is shown in FIG. 40. Proliferation was measured through flow cytometry and CFSE staining.

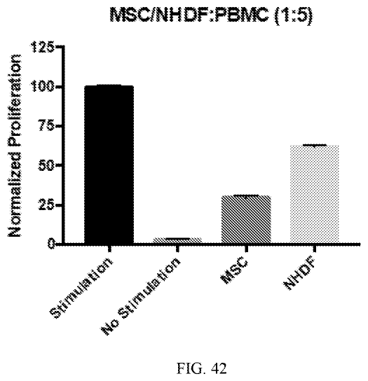

Results pertaining to dose response are shown in FIGS. 41 and 42. The MSC:PBMC ratio demonstrates a dose response like curve. NHDF demonstrates immunosuppressive ability, but less than MSCs.

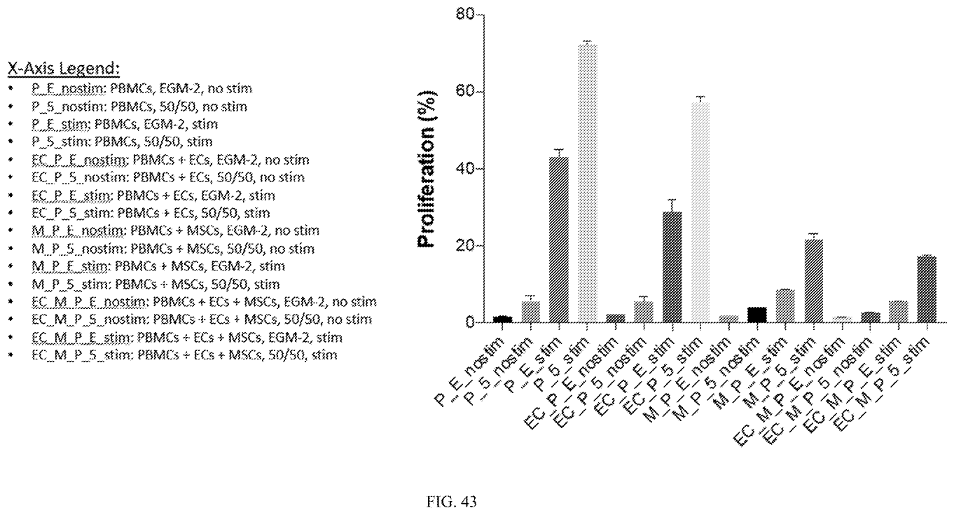

Results pertaining to co-culture effect on T-cell proliferation are shown in FIG. 43. ECs or MSCs were cultured at 150 k/well at a 1:10 ratio to PBMCs. The term EGM-2 stands for EGM-2 EC cell media. The abbreviation 50/50 stands for EGM-2+PBMC media (50/50 mix). The abbreviation EC stands for endothelial cell. The abbreviation M stands for mesenchymal stem cell. The abbreviation EC_M stands for 150 k ECS & 150 k MSCs. ECs alone show modest immunomodulatory effects. MSCs show significant immunosuppression. EC+MSC shows only slight improvement over MSC alone.

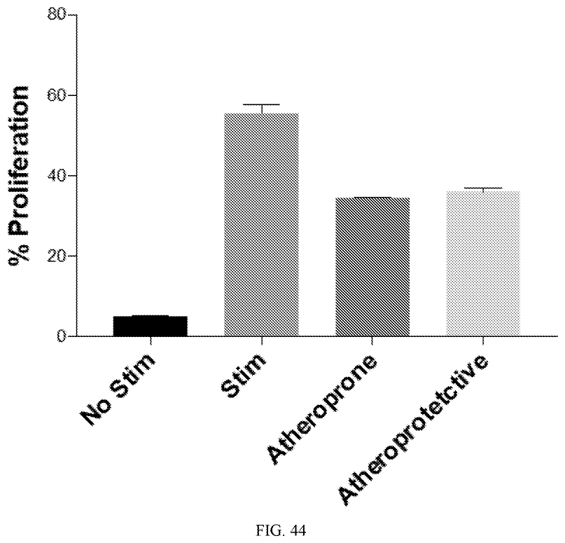

Results pertaining to the effect of EC phenotypes on T-cell proliferation are shown in FIG. 44. ECs were cultured at 150 k/well at a 1:10 ratio to PBMCs. There were no observable differences between atheroprone and atheroprotective EC phenotype. Both demonstrate immunosuppressive phenotypes.

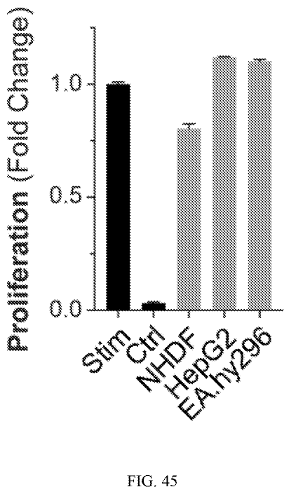

Results showing the normalized proliferative response of PBMC co-culture with NHDF (dermal fibroblast), HepG2 (liver), and EA.hy296 (endothelial) are shown in FIG. 45. Non-MSC cells show significantly reduced ability to suppress PBMC proliferation compared to MSCs. HepG2 and EA.hy296 cells interestingly show enhanced proliferation.

### REFERENCES

Aiuti, A, Biasco, L, Scaramuzza, S, Ferrua, F, Cicalese, M P, Baricordi, C et al. (2013). Lentiviral hematopoietic stem cell gene therapy in patients with Wiskott-Aldrich syndrome. Science, 341: 1233151.
Ausubel, L., Hall, C., Sharma, A., et al. (2012). Production of CGMP-Grade Lentiviral Vectors. Bioprocess Int, 10(2): 32-43.
Bhatia, M., Bonnet, D., Kapp, U., Wang, J. C. Y., Murdoch, B., and Dick, J. E. (1997). Quantitative Analysis Reveals Expansion of Human Hematopoietic Repopulating Cells After Short-term Ex Vivo Culture. The Journal of Experimental Medicine 186, 619-624.
Biffi, A, Montini, E, Lorioli, L, Cesani, M, Fumagalli, F, Plati, T et al. (2013). Lentiviral hematopoietic stem cell gene therapy benefits metachromatic leukodystrophy. Science, 341: 1233158.
Cartier, N, Hacein-Bey-Abina, S, Bartholomae, C C, Veres, G, Schmidt, M, Kutschera, I et al. (2009). Hematopoietic stem cell gene therapy with a lentiviral vector in X-linked adrenoleukodystrophy. Science, 326: 818-823.
Conneally, E., Cashman, J., Petzer, A., and Eaves, C. (1997). Expansion in vitro of transplantable human cord blood stem cells demonstrated using a quantitative assay of their lympho-myeloid repopulating activity in nonobese diabetic-scid/scid mice. Proceedings of the National Academy of Sciences 94, 9836-9841.
Coughlin, T. R., and Niebur, G. L. (2012). Fluid shear stress in trabecular bone marrow due to low-magnitude high-frequency vibration. Journal of Biomechanics 45, 2222-2229.
Davis, T. A., Wiesmann, W., Kidwell, W., Cannon, T., Kerns, L., Serke, C., Delaplaine, T., Pranger, A., and Lee, K. P. (1996). Effect of spaceflight on human stem cell hematopoiesis: suppression of erythropoiesis and myelopoiesis. Journal of Leukocyte Biology 60, 69-76.
De Leon, A., Mayani, H., and Ramirez, O. T. (1998). Design, characterization and application of a minibioreactor for the culture of human hematopoietic cells under controlled conditions. Cytotechnology 28, 127-138.
Delaney, C., Heimfeld, S., Brashem-Stein, C., Voorhies, H., Manger, R. L., and Bernstein, I. D. (2010). Notch-mediated expansion of human cord blood progenitor cells capable of rapid myeloid reconstitution. Nature medicine 16, 232-236.
Delaney, C., Varnum-Finney, B., Aoyama, K., Brashem-Stein, C., and Bernstein, I. D. (2005). Dose-dependent effects of the Notch ligand Delta1 on ex vivo differentiation and in vivo marrow repopulating ability of cord blood cells. Blood 106, 2693-2699.
Dormady, S. P., Zhang, X.-M., and Basch, R. S. (2000). Hematopoietic progenitor cells grow on 3T3 fibroblast monolayers that overexpress growth arrest-specific gene-6 (GAS6). Proceedings of the National Academy of Sciences 97, 12260-12265.
Durand, S. and Cimarelli, A. (2011). The Inside Out of Lentiviral Vectors. Viruses. 3(2): 132-159.
Farley, D C., Iqball, S., Smith, J C., Miskin, J E., Kingsman, S M., Mitrophanous, K A. (2007). Factors that influence VSV-G pseudotyping and transduction efficiency of lentiviral vectors-in vitro and in vivo implications. J Gene Med, 9(5): 345-56.
Futrega, K., Atkinson, K., Lott, W. B., and Doran, M. R. (2017). Spheroid Coculture of Hematopoietic Stem/Progenitor Cells and Monolayer Expanded Mesenchymal Stem/Stromal Cells in Polydimethylsiloxane Microwells Modestly Improves In Vitro Hematopoietic Stem/Progenitor Cell Expansion. Tissue engineering Part C, Methods 23, 200-218.
Gandara, C., Affleck, V., Stoll, E A. (2018). Manufacture of Third-Generation Lentivirus for Preclinical Use, with Process Development Considerations for Translation to Good Manufacturing Practice. Human Gene Therapy Methods, 29(1): 1-15.
Geraerts, M., Willems, S., Baekelandt, V., Debyser, Z., Gijsbers, R. (2006). Comparison of lentiviral vector titration methods. BMC Biotechnol, 6: 34.
Gori, J. L., Butler, J. M., Kunar, B., Poulos, M. G., Ginsberg, M., Nolan, D. J., Norgaard, Z. K., Adair, J. E., Rafii, S., and Kiem, H.-P. (2017). Endothelial Cells Promote Expansion of Long-Term Engrafting Marrow Hematopoietic Stem and Progenitor Cells in Primates. STEM CELLS Translational Medicine 6, 864-876.
Hacein-Bey-Abina, S., Pai, S. Y., Gaspar, H. B., Armant, M., Berry, C. C., Blanche, S., Bleesing, J., Blondeau, J., de Boer, H., Buckland, K. F. et al. (2014). A modified gamma-retrovirus vector for X-linked severe combined immunodeficiency. N. Engl. J. Med., 371, 1407-1417.
Himburg, H. A., Muramoto, G. G., Daher, P., Meadows, S. K., Russell, J. L., Doan, P., Chi, J. T., Salter, A. B., Lento, W. E., Reya, T., et al. (2010). Pleiotrophin regulates the expansion and regeneration of hematopoietic stem cells. Nature medicine 16, 475-482.
Irani, A. A., Craig, S. S., Nilsson, G., Ishizaka, T., and Schwartz, L. B. (1992). Characterization of human mast cells developed in vitro from fetal liver cells cocultured with murine 3T3 fibroblasts. Immunology 77, 136-143.
Koller, M. R., Bender, J. G., Miller, W. M., and Papoutsakis, E. T. (1993a). Expansion of primitive human hematopoietic progenitors in a perfusion bioreactor system with IL-3, IL-6, and stem cell factor. Bio/technology (Nature Publishing Company) 11, 358-363.
Koller, M. R., Emerson, S. G., and Palsson, B. O. (1993b). Large-scale expansion of human stem and progenitor cells from bone marrow mononuclear cells in continuous perfusion cultures. Blood 82, 378-384.
Kumar, S., and Geiger, H. (2017). HSC Niche Biology and HSC Expansion Ex Vivo. Trends in Molecular Medicine 23, 799-819.
Levee, M. G., Lee, G. M., Paek, S. H., and Palsson, B. O. (1994). Microencapsulated human bone marrow cultures: a potential culture system for the clonal outgrowth of hematopoietic progenitor cells. Biotechnology and bioengineering 43, 734-739.
Levi-Schaffer, F., Austen, K. F., Caulfield, J. P., Hein, A., Gravallese, P. M., and Stevens, R. L. (1987). Co-culture of human lung-derived mast cells with mouse 3T3 fibroblasts: morphology and IgE-mediated release of histamine, prostaglandin D2, and leukotrienes. Journal of immunology (Baltimore, Md.: 1950) 139, 494-500.
Liu, N., Zang, R., Yang, S.-T., and Li, Y. (2014). Stem cell engineering in bioreactors for large-scale bioprocessing. Engineering in Life Sciences 14, 4-15.
Lorenz, E., Uphoff, D., Reid, T. R., and Shelton, E. (1951). Modification of irradiation injury in mice and guinea pigs by bone marrow injections. Journal of the National Cancer Institute 12, 197-201.
Lui, W. C., Chan, Y. F., Chan, L. C., and Ng, R. K. (2014). Cytokine combinations on the potential for ex vivo expansion of murine hematopoietic stem cells. Cytokine 68, 127-132.
Meissner, P., Schroder, B., Herfurth, C., and Biselli, M. (1999). Development of a fixed bed bioreactor for the expansion of human hematopoietic progenitor cells. Cytotechnology 30, 227-234.
Merten, O., Charrier, S., Laroudie, N., et al. (2010). Large-Scale Manufacture and Characterization of a Lentiviral Vector Produced for Clinical Exx Vivo Gene Therapy Application. Human Gene Therapy, 22(3), 343-356.
Merten, O., Hebben, M., & Bovolenta, C. (2016). Production of lentiviral vectors. Molecular Therapy—Methods & Clinical Development, 3, 16017.
Milone, M. and O'Doherty, U. (2018). Clinical use of lentiviral vectors. Leukemia, 32, 1529-1541.
Mukherjee, S and Thrasher, AJ (2013). Gene therapy for PIDs: progress, pitfalls and prospects. Gene, 525: 174-181.
Obi, S., Yamamoto, K., and Ando, J. (2014). Effects of Shear Stress on Endothelial Progenitor Cells. Journal of Biomedical Nanotechnology 10, 2586-2597.
Palsson, B. O., Paek, S. H., Schwartz, R. M., Palsson, M., Lee, G. M., Silver, S., and Emerson, S. G. (1993). Expansion of human bone marrow progenitor cells in a high cell density continuous perfusion system. Bio/technology (Nature Publishing Company) 11, 368-372.
Pan, X., Sun, Q., Zhang, Y., Cai, H., Gao, Y., Shen, Y., and Zhang, W. (2017). Biomimetic Macroporous PCL Scaffolds for Ex Vivo Expansion of Cord Blood-Derived CD34+ Cells with Feeder Cells Support. Macromolecular Bioscience 17, 1700054-n/a.
Peris, P., Roforth, M. M., Nicks, K. M., Fraser, D., Fujita, K., Jilka, R. L., Khosla, S., and McGregor, U. (2015). Ability of Circulating Human Hematopoietic Lineage Negative Cells to Support Hematopoiesis. Journal of cellular biochemistry 116, 58-66.
Perucca, S., Di Palma, A., Piccaluga, P. P., Gemelli, C., Zoratti, E., Bassi, G., Giacopuzzi, E., Lojacono, A., Borsani, G., Tagliafico, E., et al. (2017). Mesenchymal stromal cells (MSCs) induce ex vivo proliferation and erythroid commitment of cord blood haematopoietic stem cells (CB-CD34+ cells). PLOS ONE 12, e0172430.
Porter, D. L., Levine, B. L., Kalos, M., Bagg, A. and June, C. H. (2011). Chimeric antigen receptor-modified T cells in chronic lymphoid leukemia. N. Engl. J. Med., 365, 725-733.
Roberts, R. A., Spooncer, E., Parkinson, E. K., Lord, B. I., Allen, T. D., and Dexter, T. M. (1987). Metabolically inactive 3T3 cells can substitute for marrow stromal cells to promote the proliferation and development of multipotent haemopoietic stem cells. Journal of cellular physiology 132, 203-214.
Rogers, G. L. and Herzog, R. W. (2015). Gene therapy for hemophilia. Front. Biosci. (Landmark Ed)., 20, 556-603.
Sandstrom, C. E., Bender, J. G., Miller, W. M., and Papoutsakis, E. T. (1996). Development of novel perfusion chamber to retain nonadherent cells and its use for comparison of human “mobilized” peripheral blood mononuclear cell cultures with and without irradiated bone marrow stroma. Biotechnology and bioengineering 50, 493-504.
Sardonini, C. A., and Wu, Y.-J. (1993). Expansion and Differentiation of Human Hematopoietic Cells from Static Cultures through Small-Scale Bioreactors. Biotechnology Progress 9, 131-137.
Schmelzer, E., Finoli, A., Nettleship, I., and Gerlach, J. C. (2015). Long-term three-dimensional perfusion culture of human adult bone marrow mononuclear cells in bioreactors. Biotechnology and bioengineering 112, 801-810.
Sheu, J., Beltzer, J., Fury, B., Wilczek, K., Tobin, S., Falconer, D., Nolta, J., Bauer, G. (2015). Large-scale production of lentiviral vector in a closed system hollow fiber bioreactor. Molecular Therapy—Methods and Clinical Development, 2, 15020.
Shpall, E. J., Quinones, R., Giller, R., Zeng, C., Baron, A. E., Jones, R. B., Bearman, S. I., Nieto, Y., Freed, B., Madinger, N., et al. Transplantation of ex vivo expanded cord blood. Biology of Blood and Marrow Transplantation 8, 368-376.
Wagner, John E., Brunstein, Claudio G., Boitano, Anthony E., DeFor, Todd E., McKenna, D., Sumstad, D., Blazar, Bruce R., Tolar, J., Le, C., Jones, J., et al. (2016). Phase I/II Trial of StemRegenin-1 Expanded Umbilical Cord Blood Hematopoietic Stem Cells Supports Testing as a Stand-Alone Graft. Cell Stem Cell 18, 144-155.
Wang, T. Y., Brennan, J. K., and Wu, J. H. (1995). Multilineal hematopoiesis in a three-dimensional murine long-term bone marrow culture. Exp Hematol 23, 26-32.
Xue, C., Kwek, K. Y. C., Chan, J. K. Y., Chen, Q., and Lim, M. (2014). The hollow fiber bioreactor as a stroma-supported, serum-free ex vivo expansion platform for human umbilical cord blood cells. Biotechnology Journal 9, 980-989.
Zandstra, P. W., Eaves, C. J., and Piret, J. M. (1994). Expansion of hematopoietic progenitor cell populations in stirred suspension bioreactors of normal human bone marrow cells. Bio/technology (Nature Publishing Company) 12, 909-914.
Zhang, C. C., Kaba, M., Ge, G., Xie, K., Tong, W., Hug, C., and Lodish, H. F. (2006). Angiopoietin-like proteins stimulate ex vivo expansion of hematopoietic stem cells. Nature medicine 12, 240-245.

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

1. A co-culture system, comprising:
a responder cell population;
a stimulator cell population, the responder and stimulator cell populations disposed within a container;
a barrier configured to physically separate the responder cell population from the stimulator cell population, the barrier being permeable to secreted factors of the stimulator cell population; and
a fluidic flow driver configured to induce a flow of a liquid suspension comprising at least one of the responder and stimulator cell populations through the container.

2. The system of claim 1, wherein the barrier is a semipermeable membrane.

3. The system of claim 1, wherein the barrier is a gel.

4. (canceled)

5. (canceled)

6. (canceled)

7. The system of claim 1, wherein the barrier comprises hollow-fiber membranes.

8. (canceled)

9. The system of claim 7, wherein the stimulator cell population is disposed within the extra luminal space of the hollow-fiber membranes at a density of about 1 to about 1,000,000 cells/cm².

10. The system of claim 1, wherein the fluidic flow driver is configured to induce a discontinuous flow of the liquid suspension.

11. (canceled)

12. (canceled)

13. (canceled)

14. (canceled)

15. (canceled)

16. The system of claim 1, wherein the responder cells are maintained at about 0.1% to about 21% partial pressure of oxygen.

17. The system of claim 1, wherein the barrier has a molecular weight cut off (MWCO) of about 30 kDA to about 100,000 kDA.

18. (canceled)

19. The system of claim 1, wherein the barrier has a pore size of about 0.00001 μm to about 0.65 μm.

20. (canceled)

21. The system of claim 1, wherein the stimulator cells are selected from the group consisting of stromal cells, viral packaging cells, antigen exposed cells, young blood cells, microbial cells, endothelial cells, fat cells, fibroblasts, cancer cells, and neurons.

22. (canceled)

23. The system of claim 1, wherein the responder cells are selected from the group consisting of peripheral blood cells, mononuclear cells, immune cells, bone marrow cells, platelets, and red blood cells.

24. (canceled)

25. (canceled)

26. The system of claim 1, wherein the secreted factors are nucleic acids.

27. (canceled)

28. The system of claim 1, wherein the secreted factors are selected from the group consisting of growth factors, chemokines, and cytokines.

29. (canceled)

30. (canceled)

31. (canceled)

32. (canceled)

33. (canceled)

34. A method of modifying cells, comprising:
exposing a responder cell population to secreted factors of a stimulator cell population, the responder and stimulator cell populations being disposed within a container and the secreted factors perfusing across a barrier separating the responder and stimulator cell populations; and
inducing a flow of a cell culture medium comprising at least one of the responder and stimulator cell populations in the container, wherein the responder cell population is modified following exposure to the secreted factors to thereby produce modified cells.

35. (canceled)

36. The method of claim 34, wherein the exposure occurs for about 1 hour to about 21 days.

37. (canceled)

38. The method of claim 34, wherein inducing the flow of the cell culture medium occurs discontinuously.

39. (canceled)

40. (canceled)

41. (canceled)

42. (canceled)

43. (canceled)

44. The method of claim 34, further comprising disposing at least one of the responder and stimulator cell populations in an intraluminal space of a hollow-fiber membrane.

45. The method of claim 34, further comprising disposing the stimulator cell population in an extraluminal space of a hollow-fiber membrane at a density of about 1 to about 1,000,000 cells/cm².

46. The method of claim 34, further comprising maintaining the responder cells at about 0.1% to about 21% partial pressure of oxygen.

47. A composition, comprising:
a population of reprogrammed cells, the reprogrammed cells:
a) including biomolecules originating from a different cell population,
b) exhibiting one or more additional or modified functional activities than a parental population of the reprogrammed cells, or
c) a combination thereof.

48-60. (canceled)
